(19) United States
(12) Patent Application Publication
(10) Pub. No.: US 2007/0172835 A1
(43) Pub. Date: Jul. 26, 2007

(54) Multiplex detection of respiratory pathogens

(75) Inventors: Mary T. McBride, Brentwood, CA (US); Thomas R. Slezak, Livermore, CA (US); James M. Birch, Albany, CA (US)

Correspondence Address: Lawrence Livermore National Laboratory, P.O. Box 808, L-703, Livermore, CA, 94551, US

(73) Assignee: The Regents of the University of California

(21) Appl. No.: 11/335,977

(22) Filed: Jan. 20, 2006

(51) Int. Cl. C12Q 1/68; C07H 21/04; C12M 1/34

(52) U.S. Cl. 435/6; 536/24.3; 435/287.2; 977/924

(57) ABSTRACT

The present invention is directed to methods, compositions and kits for multiplex detection of pathogens, such as respiratory pathogens.

### GOVERNMENT SUPPORT

The United States Government has rights in this invention pursuant to Contract No. W-7405-ENG-48 between the U.S. Department of Energy and the University of California, for the operation of Lawrence Livermore National Laboratory.

### FIELD OF THE INVENTION

The present invention is directed to methods, compositions and kits for multiplex detection of pathogens, such as respiratory pathogens.

### BACKGROUND OF THE INVENTION

During flu season, as many as half of adult patients admitted to the emergency room are admitted with respiratory complaints. Clinical samples are generally obtained as nasopharyngeal or throat swabs, nasal aspirate, or nasal washes, and are analyzed using viral culture, enzyme immunoassay (EIA), direct immunofluorescence antibody staining (DFA), or reverse transcriptase-polymerase chain reaction (RT-PCR). Viral culture (the gold standard) is both sensitive and specific, but it requires 3-10 days to provide results, far too late to establish the cause of an outbreak of respiratory illness for early intervention; the method is also labor-intensive. EIA and optical immunoassay can provide rapid results (30 minutes), but the assays lack adequate sensitivity and specificity. DFA exhibits sensitivity comparable to viral culture. DFA reagents are the mainstay of respiratory virus detection in many hospitals since reagents can detect more than one respiratory pathogen simultaneously (i.e., multiplexed) from a single sample. Multiplexed assays have been developed for detection of the most common respiratory diseases, including influenza A and B, respiratory syncytial virus (RSV), parainfluenza (Types 1-3) and adenovirus. Results can be obtained in 1-2 hours. DFA, however, requires samples with adequate numbers of target cells, high-quality equipment, a skilled microscopist, and is ultimately labor-intensive and subjective, making it less suitable for use in reference laboratories. Many groups have demonstrated that the sensitivity and specificity of RT-PCR assays for Influenza A and B are on par with viral culture and DFA; results can be obtained in 2 hours, and large numbers of samples can be rapidly tested; however, multiplexed RT-PCR assays are not available. A number of rapid diagnostic test kits for detection of influenza are commercially available (e.g., Becton-Dickenson Directagen Flu A, B-D Directagen Flu A+B, Binax NOW Flu Test, ZymeTx ZstatFlu). The rapid test kits generally provide results within 24 hours and are approximately 70% sensitive for detecting influenza and approximately 90% specific. The sensitivity of the rapid test kits means that as many as 30% of samples may yield false negatives, and the tests are not multiplexed. Each of these assay techniques has advantages and disadvantages that make them more or less suitable for use in public health laboratories, or hospital-based laboratories, but none of these existing assays are currently employed at point-of care: They all conducted in a laboratory and usually results are not produced rapidly enough to impact on the prescribed treatment.

Accordingly, there exists a significant need for rapid and accurate multiplex tests for identification of respiratory pathogens.

### SUMMARY OF THE INVENTION

The invention is directed generally to a composition comprising nucleic acids that are identified in SEQ ID NOs 1 through 74 that are specific to various respiratory pathogens.

In addition, the invention is directed to a method of detecting various respiratory pathogens from a sample by using the nucleic acids identified in SEQ ID NOs 1 through 74.

In addition, the invention is directed to a kit for the detection of various respiratory pathogens that comprises using the nucleic acids that are identified in SEQ ID NOs 1 through 74.

Also included in the invention is a composition comprising nucleic acids that are identified in SEQ ID NOs 3, 6, 9, 12, 15, 18, 21, 24, 27, 30, 33, 36, 39, 42, 45, 48, 51, 54, 57, 60, 63, 66, 69, 72 and 75.

### BRIEF DESCRIPTION OF THE DRAWINGS

FIG. 1 is a fluidic diagram that illustrates one embodiment of a nucleic acid analyzer of the present invention.

FIG. 2 shows additional details of the reagent delivery system of the hybrid nucleic acid analyzer of FIG. 1.

FIG. 3 shows additional details of the thermal cycler of the hybrid nucleic acid analyzer of FIG. 1.

FIG. 4 shows additional details of the flow cytometer of the hybrid nucleic acid analyzer of FIG. 1.

FIG. 5 shows exemplary beads used in the hybridization chamber and flow cytometer of FIG. 1.

FIG. 6 illustrates how the beads are used in the hybridization chamber and the flow cytometer described in FIG. 1.

FIG. 7 illustrates how the beads are analyzed in the flow cytometer of the system.

FIG. 8 illustrates an overview of the assay development process.

FIG. 9 illustrates a preferred fluidics manifold.

### DETAILED DESCRIPTION OF THE PREFERRED EMBODIMENT

Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

The term “pathogen” means any disease-producing agent (especially a virus or bacterium or other microorganism).

The term “respiratory pathogen” means a pathogen capable of infecting respiratory tracts, of humans, in particular.

Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As noted above, it is clear that respiratory pathogens pose an increasingly viable threat. Given the multitude of potential respiratory pathogens, there is an increasing demand for rapid, sensitive and accurate tests to detect a multiple pathogenic organisms.

Accordingly, nucleic acid-based assays have been developed that are rapid, sensitive, specific and can detect a plurality of different respiratory pathogens simultaneously from a single sample. For example, the multiplexed assays have been developed for FluIDₓ, an integrated system designed for use at point-of-care. FluIDₓ is capable of sample preparation and processing (e.g., nucleic acid extraction), performing highly multiplexed real-time PCR nucleic-acid based assays, data analysis and reporting, and system decontamination between samples, where all functions are completely automated. FluIDₓ currently employs a 27-plex assay, comprising assays for 8 respiratory pathogens (influenza A and B, parainfluenza Types 1-3, respiratory syncytial virus, adenovirus, and SARS) where each agent is represented by multiple loci, and 5 unique internal controls. Results on a patient sample can be provided in about 2 hours

Accordingly, the present invention provides rapid, sensitive and accurate tests to detect respiratory organisms. In particular, the invention provides signature sequences of various respiratory organisms that find use in detecting the presence of a particular organism.

In addition, the present invention provides primers capable of detecting and/or amplifying the signature sequence or sequences of the organism.

While the present method finds use in detecting any pathogen or target sequence, preferred respiratory pathogens include, but are not limited to, influenza A, influenza B, parainfluenza, respiratory syncytial virus, adenovirus, picornavirus and the like. Others include SARs (coranavirus). Additionally, the same methods may be used for the detection of bacterial pathogens, and bacterial respiratory pathogens. Bacterial respiratory pathogens include things like pneumococcus, streptococcus, and staphylococuus.

The present invention provides a method that includes amplifying target specific nucleic acids with primers capable of amplifying the signature sequence or sequences of the target organism. Following amplification, the amplicon is detected. By “amplicon” is meant the amplified product of a nucleic acid amplification reaction. The presence of a signature sequence indicates the presence of the respiratory organism in the sample. Amplicons are detected by any of a variety of methods that are known in the art but preferably detection occurs via a flow cytometer, e.g. Luminex detector.

Accordingly, the present invention provides compositions and methods for detecting the presence or absence of respiratory pathogen target nucleic acid sequences in a sample. As will be appreciated by those in the art, the sample solution may comprise any number of things, including, but not limited to, bodily fluids (including, but not limited to, blood, nasopharyngeal secretions, urine, serum, lymph, saliva, anal and vaginal secretions, perspiration and semen, of virtually any organism, with mammalian samples being preferred, environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.; As will be appreciated by those in the art, virtually any experimental manipulation may have been done on the sample.

The compositions and methods of the invention are directed to the detection of target sequences. The term “target sequence” or “target nucleic acid” or grammatical equivalents herein means a nucleic acid sequence on a single strand of nucleic acid or its complement. The target sequence may be a portion of a gene, a regulatory sequence, genomic DNA, cDNA, RNA including mRNA and rRNA, or others.

Generally, as outlined herein, a target sequence from a sample is amplified to produce a secondary target, e.g. the amplicon, that is detected, as outlined herein. Alternatively, an amplification step is done using a signal probe that is amplified, again producing a secondary target that is detected. The target sequence may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

As is outlined more fully below, probes are made to hybridize to target sequences to determine the presence or absence of the target sequence in a sample. Also, primers are made to amplify target sequences to determine the presence or absence of the target sequence in a sample. Generally speaking, this term will be understood by those skilled in the art.

If required, the target sequence is prepared using known techniques. For example, the sample may be treated to lyse the cells, using known lysis buffers, sonication, electroporation, etc., with purification occurring as needed, as will be appreciated by those in the art. In addition, the reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents which may be included in the assays. These include reagents like salts, buffers, neutral proteins, e.g. albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

In addition, in most embodiments, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques may also be used.

A primer nucleic acid is then contacted to the target sequence to form a hybridization complex. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally performed under stringency conditions that allow formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The size of the primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on the use and amplification technique.

In addition, the different amplification techniques may have further requirements of the primers, as is more fully described below.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identity of the enzyme will depend on the amplification technique used, as is more fully outlined below. Similarly, the modification will depend on the amplification technique, as outlined below.

Once the enzyme has modified the primer to form a modified primer, the hybridization complex is disassociated. In one aspect, dissociation is by modification of the assay conditions. In another aspect, the modified primer no longer hybridizes to the target nucleic acid and dissociates. Either one or both of these aspects can be employed in signal and target amplification reactions as described below. Generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

After a suitable time of amplification, unreacted primers are removed, in a variety of ways, as will be appreciated by those in the art and described below, and the hybridization complex is disassociated. In general, the modified primer comprises a detectable label, such as a fluorescent label, which is either incorporated by the enzyme or present on the original primer, and the modified primer then detected.

Probes/Primers

As one of skill in the art appreciates, there are several probes or primers that are used in the present invention. These probes/primers can take on a variety of configurations and may have a variety of structural components described in more detail below.

Generally the probe/primer includes target pathogen specific sequences sufficient to confer specific amplification or hybridization to the respective respiratory DNA. In addition, at least one primer of a primer pair contains an adapter.

The size of the primer and probe nucleic acid may vary, as will be appreciated by those in the art with each portion of the probe and the total length of the probe in general varying from 5 to 500 nucleotides in length. Each portion is preferably between 10 and 100 being preferred, between 15 and 50 being particularly preferred, and from 10 to 35 being especially preferred, depending on the use and amplification technique. The adapter sequences of the probes are preferably from 15-25 nucleotides in length, with 20 being especially preferred. The target specific portion of the probe is preferably from 15-50 nucleotides in length. In addition, the primer may include one or more additional amplification priming sites.

In a preferred embodiment, the target specific probe or probes comprises a target domain substantially complementary to a first domain of the target sequence. In general, probes of the present invention are designed to be complementary to a target sequence (either the target sequence of the sample or to other probe sequences, as is described herein), such that hybridization of the target and the probes of the present invention occurs.

In a preferred embodiment, one of the probes further comprises an adapter sequence, (sometimes referred to in the art as “zip codes” or “bar codes”). Adapters facilitate immobilization of probes to solid supports. That is, arrays (either solid phase or liquid phase arrays) are generated that contain capture probes that are not target specific, but rather specific to individual (preferably) artificial adapter sequences.

Thus, an “adapter sequence” is a nucleic acid that is generally not native to the target sequence, i.e. is exogenous, but is added or attached to the target sequence. It should be noted that in this context, the “target sequence” can include the primary sample target sequence, or can be a derivative target such as a reactant or product of the reactions outlined herein; thus for example, the target sequence can be a PCR product, a first ligation probe or a ligated probe in an OLA reaction, etc. The terms “barcodes”, “adapters”, “addresses”, “tags” and “zipcodes” have all been used to describe artificial sequences that are added to amplicons to allow separation of nucleic acid fragment pools. One preferred form of adapters are hybridization adapters. In this embodiment adapters are chosen so as to allow hybridization to the complementary capture probes on a surface of an array. Adapters serve as unique identifiers of the probe and thus of the target sequence. In general, sets of adapters and the corresponding capture probes on arrays are developed to minimize cross-hybridization with both each other and other components of the reaction mixtures, including the target sequences and sequences on the larger nucleic acid sequences outside of the target sequences (e.g. to sequences within genomic DNA). Preferred adapters are those that meet the following criteria. They are not found in the genome of the target organism and they do not have undesirable structures, such as hairpin loops.

As will be appreciated by those in the art, the attachment, or joining, of the adapter sequence to the target sequence can be done in a variety of ways. In a preferred embodiment, the adapter sequences are added to the primers of the reaction (extension primers, amplification primers, readout probes, genotyping primers, Rolling Circle primers, etc.) during the chemical synthesis of the primers. The adapter then gets added to the reaction product during the reaction; for example, the primer gets extended using a polymerase to form the new target sequence that now contains an adapter sequence. Alternatively, the adapter sequences can be added enzymatically. Furthermore, the adapter can be attached to the target after synthesis; this post-synthesis attachment can be either covalent or non-covalent. In a preferred embodiment the adapter is added to the target sequence or associated with a particular allele during an enzymatic step.

In addition, as will be appreciated by those in the art, the adapter can be attached either on the 3′ or 5′ ends, or in an internal position, depending on the configuration of the system.

In one embodiment the use of adapter sequences allow the creation of more “universal” surfaces; that is, one standard array, comprising a finite set of capture probes can be made and used in any application. The end-user can customize the array by designing different soluble target probes, which, as will be appreciated by those in the art, is generally simpler and less costly. In a preferred embodiment, an array of different and usually artificial capture probes are made; that is, the capture probes do not have complementarity to known target sequences. The adapter sequences can then be incorporated in the target probes.

As will be appreciated by those in the art, the length of the adapter sequences will vary, depending on the desired “strength” of binding and the number of different adapters desired. In a preferred embodiment, adapter sequences range from about 6 to about 500 basepairs in length, with from about 8 to about 100 being preferred, and from about 10 to about 25 being particularly preferred.

In a preferred embodiment, the adapter sequence uniquely identifies the target analyte, e.g. respiratory organism nucleic acid, to which the target probe binds. That is, while the adapter sequence need not bind itself to the target analyte, the system allows for identification of the target analyte by detecting the presence of the adapter. Accordingly, following a binding or hybridization assay and washing, the probes including the adapters are amplified. Detection of the adapter then serves as an indication of the presence of the target analyte.

SIn a preferred embodiment target specific sequences from the SARS virus, influenza, RSV, Adenovirus and parainfluenza include the primers and probes as set forth in Table 1.

Preferably a detection assay includes the use of each of the PCR primers listed for the respective pathogen. However, in some embodiments fewer of all of the PCR primers can be used. When fewer of the PCR primers are used, at least two pairs are used and the use of at least 3 or 4 or 5 or 6 or 7 or 8 or 9 or 10 or 11 PCR primer pairs are used. Accordingly, the apparatus of the invention includes such PCR primers for amplification of the respective pathogen sequence. Also, the use of each probe listed for the respective pathogen is preferred, although in some embodiments, fewer than all of the probes are used.

Accordingly, in a preferred embodiment the invention provides the use of all PCR primers in Table 1. Alternatively, the invention provides the use of all PCR primers for a particular pathogen. In yet another embodiment the invention provides the use of all of the probes in Table 1. Alternatively, the invention provides the use of all probes in Table 1.

TABLE 1
Components of the Multiplexed Assay Panel for Detection of Respiratory Pathogens

| | Target | | | |
|---|---|---|---|---|
| Identification | Gene | Forward Sequence | Reverse Sequence | Probe Sequence |
| Influenza A | H1 | AGCGTCAAAAATGGGACTTATGA | AAAGACCCATTAGAGCACATCCA | TGGCGATCTATTCAACTGTCGCCA |
| 000022_1 | | (SEQ ID NO: 1) | (SEQ ID NO: 2) | GTT |
| | | | | (SEQ ID NO: 3) |
| Influenza A | H1 | CTTTCAGCTACAGATGCAGACACA | TTCCCATTGTGACTGTCCTCAA | CGAACAATTCAACCGACACTGTTG |
| 000001_1 | | (SEQ ID NO: 4) | (SEQ ID NO: 5) | ACACA |
| | | | | (SEQ ID NO: 6) |
| Influenza A | H1 | GCCATTAACGGGATTACAAACAAG | CCAGTAGAACCAACAATTCTGCA | TCGAGAAAATGAACACTCAATTCA |
| 000041_1 | | (SEQ ID NO: 7) | ATTCTGCATTAT | CAGCTGTG |
| | | | (SEQ ID NO: 8) | (SEQ ID NO: 9) |
| Influenza A | H3 | ATGCTGAGGATATGGGCAATG | GATATGGCAAAGGAAATCCATAGG | CATTAAACAACCGGTTCCAGATCA |
| 008182_1 | | (SEQ ID NO: 10) | (SEQ ID NO: 11) | AAGGTGT |
| | | | | (SEQ ID NO: 12) |
| Influenza A | H3 | TATCACAAATGTGATAATGCATGC | ATGAAACCCAATAGAACAACACAA | TGTGGATTTCATTCGCCATATCAT |
| 000031_1 | | A | AT | GCTTC |
| | | (SEQ ID NO: 13) | (SEQ ID NO: 14) | (SEQ ID NO: 15) |
| Influenza A | H5 | GGGAGGAAATAGACGGAGTCAAA | TAGATGCAAATTCTGCACTGCAA | TCAACAGTGGCGAGTTCCCTAGCA |
| 009339_1 | | (SEQ ID NO: 16) | (SEQ ID NO: 17) | CTG |
| | | | | (SEQ ID NO: 18) |
| Influenza A | H5 | GTATGGGTACCACCATAGCCAATG | TGTTCATTTTGTCAATGATCGAGT | TGCAGACAAAGAATCCACTCAAAA |
| 000772_1 | | A | T | GGCAA |
| | | (SEQ ID NO: 19) | (SEQ ID NO: 20) | (SEQ ID NO: 21) |
| Influenza A | H5 | GATCTAAATGGAGTGAAGCCTCTC | TATGTAAGACCATTCCGGCACAT | CTGGATGGCTCCTCGGAAACCCTA |
| 006709_1 | | AT | (SEQ ID NO: 23) | TGT |
| | | (SEQ ID NO: 22) | | (SEQ ID NO: 24) |
| Influenza A | H5 | GACAATGAATGTATGGAAAGTGTG | ATCCAAAAAGATAGACCAGCTATC | CAGTGGCAAGTTCCCTAGCACTGG |
| 011991_1 | | AGA | ATG | CA |
| | | (SEQ ID NO: 25) | (SEQ ID NO: 26) | (SEQ ID NO: 27) |
| Influenza A | H7 | GATCCCAATGACACAGTGACCTT | TTCCCCACAGTTCTAGGGTTGA | CATAGCCCCTGACAGGGCAAGTTT |
| 000025_1 | | (SEQ ID NO: 28) | (SEQ ID NO: 29) | CTTTAG |
| | | | | (SEQ ID NO: 30) |
| Influenza A | H7 | GGCTACAAAGATGTGATACTTTGG | CATGTTTCCATTCTTCACACACAT | CTTCTGGCCATTGCAATGGGCC |
| 000049_1 | | TTT | (SEQ ID NO: 32) | (SEQ ID NO: 33) |
| | | (SEQ ID NO: 31) | | |
| Influenza A | H7 | TAAGCAGCGGCTACAAAGATGT | GCACCGCATGTTTCCATTT | CATGTTTCATACTTCTGGCCATTG |
| 000403_1 | | (SEQ ID NO: 34) | (SEQ ID NO: 35) | CAATGG |
| | | | | (SEQ ID NO: 36) |
| RSV 1769653 | M HRSVgp05 | AATGCTATCACCAATGCGAAAA | AACGTGTAGCTGTATGCTTCCAA | TGACAATAAAGGAGCATTCAAATA |
| 1769653 | matrix | (SEQ ID NO: 37) | (SEQ ID NO: 38) | TATCAAGCCACA |
| | protein | | | (SEQ ID NO: 39) |
| Adenovirus B | E4 | CGCTTTCACAGTCCAACTGC | GCTGCTTGTGGGTTTGATGA | CGTTTTCGGATTATGATTCCCATC |
| 1770195 | HAdVBgp12 | (SEQ ID NO: 40) | (SEQ ID NO: 41) | GTTCTTC |
| | | | | (SEQ ID NO: 42) |
| Adenovirus B | L5 | TCCTGCACCATTCCCAGATA | CCTCCGGGACCTGTTTGTAA | CAGCTTTCCAGCCTTGAATTATTC |
| 1770201 | HAdVBgp11 | (SEQ ID NO: 43) | (SEQ ID NO: 44) | GTGTCAG |
| | agnoprotein | | | (SEQ ID NO: 45) |
| Adenovirus C | intergenic | AGCGCGTAATATTTGTCTAGGGC | TCAGCTGACTATAATAATAAAACG | CGGAACGCGGAAAACACCTGAGAA |
| 1768012 | (at base | (SEQ ID NO: 46) | CCA | AA |
| | 331) | | (SEQ ID NO: 47) | (SEQ ID NO: 48) |
| Adenovirus C | intron of | TCGATCTTACCTGCCACGAG | GCCACAGGTCCTCATATAGCAA | TGCTCCACATAATCTAACACAAAC |
| 1768014 | E1A (at | (SEQ ID NO: 49) | (SEQ ID NO: 50) | TCCTCACCC |
| | base 917) | | | (SEQ ID NO: 51) |
| Adenovirus C | pV/minor | CCCAACACCTAGCCTAAAGCC | TTTCCAAGACATCTTCCAGTCG | AAGTCACCAGACTCGCGCTTTAGG |
| 1768040 | core protein | (SEQ ID NO: 52) | (SEQ ID NO: 53) | CC |
| | | | | (SEQ ID NO: 54) |
| Adenovirus D | HAdVDgp20 | TGGTCCAGATGGAAAGGTCA | CTTTTGCTGTTGCCTCTGTCA | TGTCACACTTACACCCTAACTTAT |
| 1768089 | predicted | (SEQ ID NO: 55) | (SEQ ID NO: 56) | ACCCAGGCTCA |
| | glycoprotein | | | (SEQ ID NO: 57) |
| Adenovirus D | HAdVDgp21 | GCGTTCTGATTAGCATAGTCACAC | GCATTTGTATGCAGTAACATTCCA | TTGTCCATGTAGTTTGTGGATAAG |
| 1768091 | hypothetical | T | (SEQ ID NO: 59) | TCCCATTCA |
| | protein | (SEQ ID NO: 58) | | (SEQ ID NO: 60) |
| Adenovirus E | L4 pVIII | GCATCGGCACTCTCCAGTT | CACCATGGGACATTCAATCG | AGTTCACTCCCTCGGTCTACTTCA |
| 1759552 | | (SEQ ID NO: 61) | (SEQ ID NO: 62) | ACCCCTT |
| | | | | (SEQ ID NO: 63) |
| Adenovirus E | HAdVEgp14 | TGCAATTTTGTTGGGTTTCG | CCTGGCTGTTATTTTCCACCA | TTAATCATGGTTCTTCCTGTTCTT |
| 1759558 | E4 orf 4 | (SEQ ID NO: 64) | (SEQ ID NO: 65) | CCCTCCC |
| | | | | (SEQ ID NO: 66) |
| Parainfluenza1 | Hpv1gp08 | TGGCTAATTGCATTGCATCC | CTCGTCCCCTTTTATTGGCA | ACATGCGGGACAAACAGAATACCA |
| 1770236 | fusion | (SEQ ID NO: 67) | (SEQ ID NO: 68) | GTGAATC |
| | glycoprotein | | | (SEQ ID NO: 69) |
| Parafinfluenza2 | L large | TGTCAAGTAATTGCGGAAGCA | GCCAATTTGACTCATAGTAAGCAA | AAGACAACTCCGTTTTCCTTCATT |
| 1770386 | protein | (SEQ ID NO: 70) | TG | AGAGTACCTGC |
| | | | (SEQ ID NO:71) | (SEQ ID NO: 72) |
| Parainfluenza3 | HPIV3gp6 | CAACGGAATGCTGTTCAATACAA | TCTTCTAGATCTGATTTGGCCTTG | TGAGCTCGATTGATATGTCAATTG |
| 1770275 | protein | (SEQ ID NO: 73) | (SEQ ID NO: 74) | GATCAAGTG |
| | | | | (SEQ ID NO: 75) |

Amplification

Target Amplification

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), transcription mediated amplification (TMA) and nucleic acid sequence based amplification (NASBA).

Polymerase Chain Reaction Amplification

In a preferred embodiment, the target amplification technique is PCR. The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtraction”, “allele-specific PCR”, among others.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer with dNTPs, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostable polymerase such as Taq I polymerase and thermal cycling.

Accordingly, the PCR reaction requires at least one PCR primer, a polymerase, and a set of dNTPs. As outlined herein, the primers may comprise the label, or one or more of the dNTPs may comprise a label.

Strand Displacement Amplification (SDA)

In a preferred embodiment, the target amplification technique is SDA. Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby expressly incorporated by reference in their entirety.

Nucleic Acid Sequence Based Amplification (NASBA) and Transcription Mediated Amplification (TMA)

In a preferred embodiment, the target amplification technique is nucleic acid sequence based amplification (NASBA). NASBA is generally described in U.S. Pat. No. 5,409,818; Sooknanan et al., Nucleic Acid Sequence-Based Amplification, Ch. 12 (pp. 261-285) of Molecular Methods for Virus Detection, Academic Press, 1995; and “Profiting from Gene-based Diagnostics”, CTB International Publishing Inc., N.J., 1996, all of which are incorporated by reference. NASBA is very similar to both TMA and QBR. Transcription mediated amplification (TMA) is generally described in U.S. Pat. Nos. 5,399,491, 5,888,779, 5,705,365, 5,710,029, all of which are incorporated by reference. The main difference between NASBA and TMA is that NASBA utilizes the addition of RNAse H to effect RNA degradation, and TMA relies on inherent RNAse H activity of the reverse transcriptase.

Signal Amplification Techniques

In a preferred embodiment, the amplification technique is signal amplification. Signal amplification involves the use of limited number of target molecules as templates to either generate multiple signaling probes or allow the use of multiple signaling probes. Signal amplification strategies include LCR, CPT, Q.beta.R, invasive cleavage technology, and the use of amplification probes in sandwich assays.

Single Base Extension (SBE)

In a preferred embodiment, single base extension (SBE; sometimes referred to as “minisequencing”) is used for amplification. Briefly, SBE is a technique that utilizes an extension primer that hybridizes to the target nucleic acid. A polymerase (generally a DNA polymerase) is used to extend the 3′ end of the primer with a nucleotide analog labeled a detection label as described herein. Based on the fidelity of the enzyme, a nucleotide is only incorporated into the extension primer if it is complementary to the adjacent base in the target strand. Generally, the nucleotide is derivatized such that no further extensions can occur, so only a single nucleotide is added. However, for amplification reactions, this may not be necessary. Once the labeled nucleotide is added, detection of the label proceeds as outlined herein. See generally Sylvanen et al., Genomics 8:684-692 (1990); U.S. Pat. Nos. 5,846,710 and 5,888,819; Pastinen et al., Genomics Res. 7(6):606-614 (1997); all of which are expressly incorporated herein by reference.

Oligonucleotide Ligation Amplification (OLA)

In a preferred embodiment, the signal amplification technique is OLA. OLA, which is referred to as the ligation chain reaction (LCR) when two-stranded substrates are used, involves the ligation of two smaller probes into a single long probe, using the target sequence as the template. In LCR, the ligated probe product becomes the predominant template as the reaction progresses. The method can be run in two different ways; in a first embodiment, only one strand of a target sequence is used as a template for ligation; alternatively, both strands may be used. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; WO 97/31256; and WO 89/09835, and U.S. Ser. Nos. 60/078,102 and 60/073,011, all of which are incorporated by reference.

Rolling-Circle Amplification (RCA)

In a preferred embodiment the signal amplification technique is RCA. Rolling-circle amplification is generally described in Baner et al. (1998) Nuc. Acids Res. 26:5073-5078; Barany, F. (1991) Proc. Natl. Acad. Sci. USA 88:189-193; and Lizardi et al. (1998) Nat. Genet. 19:225-232, all of which are incorporated by reference in their entirety.

In general, RCA may be described in two ways. First, as is outlined in more detail below, a single probe is hybridized with a target nucleic acid. Each terminus of the probe hybridizes adjacently on the target nucleic acid and the OLA assay as described above occurs. When ligated, the probe is circularized while hybridized to the target nucleic acid. Addition of a polymerase results in extension of the circular probe. However, since the probe has no terminus, the polymerase continues to extend the probe repeatedly. Thus results in amplification of the circular probe.

A second alternative approach involves OLA followed by RCA. In this embodiment, an immobilized primer is contacted with a target nucleic acid. Complementary sequences will hybridize with each other resulting in an immobilized duplex. A second primer is contacted with the target nucleic acid. The second primer hybridizes to the target nucleic acid adjacent to the first primer. An OLA assay is performed as described above. Ligation only occurs if the primer are complementary to the target nucleic acid. When a mismatch occurs, particularly at one of the nucleotides to be ligated, ligation will not occur. Following ligation of the oligonucleotides, the ligated, immobilized, oligonucleotide is then hybridized with an RCA probe. This is a circular probe that is designed to specifically hybridize with the ligated oligonucleotide and will only hybridize with an oligonucleotide that has undergone ligation. RCA is then performed as is outlined in more detail below.

Accordingly, in a preferred embodiment, a single oligonucleotide is used both for OLA and as the circular template for RCA (referred to herein as a “padlock probe” or a “RCA probe”). That is, each terminus of the oligonucleotide contains sequence complementary to the target nucleic acid and functions as an OLA primer as described above. That is, the first end of the RCA probe is substantially complementary to a first target domain, and the second end of the RCA probe is substantially complementary to a second target domain, adjacent to the first domain. Hybridization of the oligonucleotide to the target nucleic acid results in the formation of a hybridization complex. Ligation of the “primers” (which are the discrete ends of a single oligonucleotide) results in the formation of a modified hybridization complex containing a circular probe i.e. an RCA template complex. That is, the oligonucleotide is circularized while still hybridized with the target nucleic acid. This serves as a circular template for RCA. Addition of a polymerase to the RCA template complex results in the formation of an amplified product nucleic acid. Following RCA, the amplified product nucleic acid is detected. This can be accomplished in a variety of ways; for example, the polymerase may incorporate labeled nucleotides, or alternatively, a label probe is used that is substantially complementary to a portion of the RCA probe and comprises at least one label is used.

The polymerase can be any polymerase, but is preferably one lacking 3′ exonuclease activity (3′ exo.sup.-). Examples of suitable polymerase include but are not limited to exonuclease minus DNA Polymerase I large (Klenow) Fragment, Phi29 DNA polymerase, Taq DNA Polymerase and the like. In addition, in some embodiments, a polymerase that will replicate single-stranded DNA (i.e. without a primer forming a double stranded section) can be used.

In a preferred embodiment, the RCA probe contains an adapter sequence as outlined herein, with adapter capture probes on the array. Alternatively, unique portions of the RCA probes, for example all or part of the sequence corresponding to the target sequence, can be used to bind to a capture probe.

Labeling Techniques

In general, either direct or indirect detection of the target products can be done. “Direct” detection as used in this context, as for the other amplification strategies outlined herein, requires the incorporation of a label, in this case a detectable label, preferably an optical label such as a fluorophore, into the target sequence, with detection proceeding as outlined below. In this embodiment, the label(s) may be incorporated in three ways: (1) the primers comprise the label(s), for example attached to the base, a ribose, a phosphate, or to analogous structures in a nucleic acid analog; (2) modified nucleosides are used that are modified at either the base or the ribose (or to analogous structures in a nucleic acid analog) with the label(s); these label-modified nucleosides are then converted to the triphosphate form and are incorporated into the newly synthesized strand by a polymerase; (3) modified nucleotides are used that comprise a functional group that can be used to add a detectable label; or (4) modified primers are used that comprise a functional group that can be used to add a detectable label. Any of these methods result in a newly synthesized strand that comprises labels, that can be directly detected as outlined below.

Thus, the modified strands comprise a detection label. By “detection label” or “detectable label” herein is meant a moiety that allows detection. This may be a primary label or a secondary label.

In a preferred embodiment, the detection label is a primary label. A primary label is one that can be directly detected, such as a fluorophore. In general, labels fall into three classes: a) isotopic labels, which may be radioactive or heavy isotopes; b) magnetic, electrical, thermal labels; and c) colored or luminescent dyes. Labels can also include enzymes (horseradish peroxidase, etc.) and magnetic particles. Preferred labels include chromophores or phosphors but are preferably fluorescent dyes. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue.™, Texas Red, alexa dyes, phycoerythin, bodipy, and others described in the 6th Edition of the Molecular Probes Handbook by Richard P. Haugland, hereby expressly incorporated by reference.

In a preferred embodiment, a secondary detectable label is used. Accordingly, detection labels may be primary labels (i.e. directly detectable) or secondary labels (indirectly detectable). A secondary label is one that is indirectly detected; for example, a secondary label can bind or react with a primary label for detection, or may allow the separation of the compound comprising the secondary label from unlabeled materials, etc. Secondary labels find particular use in systems requiring separation of labeled and unlabeled probes, such as SBE reactions. Secondary labels include, but are not limited to, one of a binding partner pair; chemically modifiable moieties; nuclease inhibitors, etc.

In a preferred embodiment, the secondary label is a binding partner pair. For example, the label may be a hapten or antigen, which will bind its binding partner. In a preferred embodiment, the binding partner can be attached to a solid support to allow separation of extended and non-extended primers. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the extension primer.

In a preferred embodiment, the binding partner pair comprises biotin or imino-biotin and streptavidin. Imino-biotin is particularly preferred as imino-biotin disassociates from streptavidin in pH 4.0 buffer while biotin requires harsh denaturants (e.g. 6 M guanidinium HCl, pH 1.5 or 90% formamide at 95° C.).

In a preferred embodiment, the binding partner pair comprises a primary detection label (for example, attached to the NTP and therefore to the extended primer) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

DetectionArrays

Detection of the amplified products described above preferably employs arrays, as defined herein.

By “substrate” or “solid support” or other grammatical equivalents herein is meant any material to which a capture probe can be immobilized. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, and a variety of other polymers. In general, the substrates allow optical detection and do not themselves appreciably fluorescese.

Generally the substrate is flat (planar), although as will be appreciated by those in the art, other configurations of substrates may be used as well.

In a preferred embodiment the array is a liquid array. In this embodiment, a species of capture probes is immobilized to a first set of microspheres. Likewise, a second species of capture probes is immobilized to a second set of microspheres. Similarly additional species of capture probes are attached to discrete populations of microspheres. There is no upward limit to the number of populations of microspheres or capture probes when populations are analyzed individually. When multiple sets of microspheres are mixed and analyzed the number of sets is limited only by the number of encoding moieties applied to the microspheres. That is, microspheres are encoded so that the identity of each set of microspheres can be determined.

Encoding moieties can be any distinguishable characteristic, e.g. size, shape, texture etc., of the microsphere. In preferred embodiments, encoding moieties are attribures that are not inherent in the bead or microsphere itself. Rather, the encoding moiety is a feature that is added to a bead. Preferred encoding moieties include, but are not limited to nucleic acids, proteins, and detectable labels or fluors. In addition, materials such as nanocrystals can be used as encoding moieties.

Also, in some embodiments, a plurality of different types of encoding moieties can be used to develop numerous different codes.

In a preferred embodiment, the beads and encoding system are those used in the Luminex flow cytometer. This system is described in more detail in U.S. Pat. No. 5,981,180, which is expressly incorporated herein by reference.

In a particularly preferred embodiment, the apparatus of the invention is an integrated system that includes an in-line thermal cycler and flow cytometer. Other components of the system are set forth in US PGPUB 2005/0239192, which is expressly incorporated herein by reference.

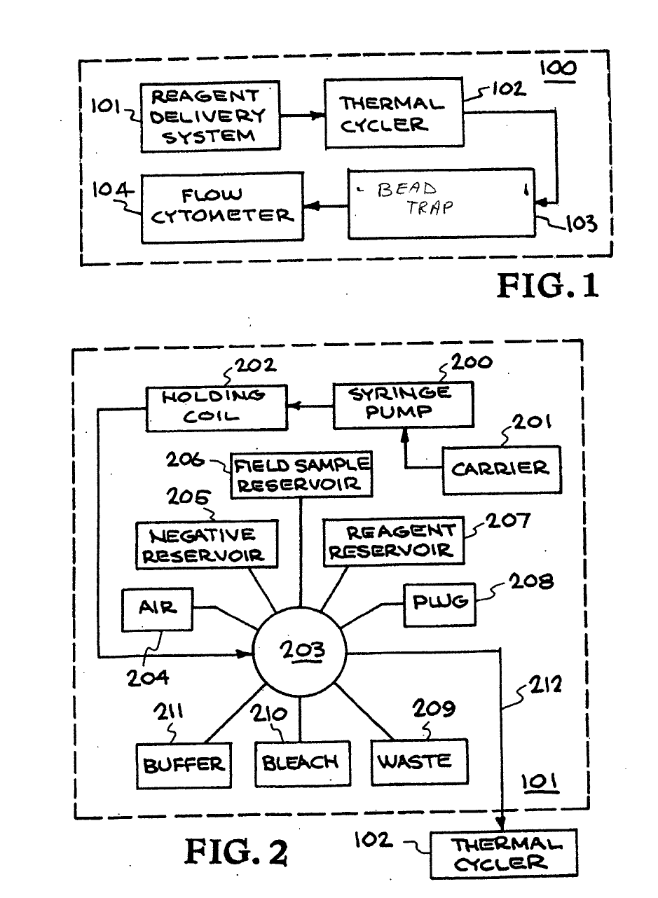

A preferred apparatus of the invention is shown in FIG. 1. FIG. 1 depicts a fluidic diagram of one embodiment of a hybrid nucleic acid analyzer. This embodiment of the hybrid nucleic acid analyzer is designated generally by the reference numeral 100. Nucleic acid and protein analyses are usually performed on separate analysis platforms.

The hybrid nucleic acid analyzer system 100 comprises a reagent delivery system 101, a thermal cycler 102, optionally a bead trap for washing 103, and a flow cytometer 104. In a particularly preferred embodiment the system does not include a hybridization chamber.

Although the prior art recognized the desire to include a separate hybridization chamber for hybridization of beads to the amplicons, the present invention performs this hybridization in the thermal cycler itself. That is, following amplification the microspheres are brought into the thermal cycler for hybridization with the amplicons. The advantage of doing so is that a separate component is, e.g. the hybridization chamber, is eliminated from the apparatus. This results in a less complicated system that provides for miniaturization. In addition, having fewer chambers in the system results in increased efficiency as compared to the apparatus with the hybridization chamber. That is, it has been found by the present invention that having fewer components allows for maximal sample recovery at each step because there are fewer manipulations with the sample and the sample is contacted with fewer surfaces. In addition, because of the improved recovery, the system of the invention lacking the hybridization chamber allows for increased signal-to-noise ratios. Without being bound by theory, it is thought that the improved recovery improves the overall signal strength.

In some embodiments, when desirable, an in-line bead-trap 103 follows the thermal cycler. In this embodiment the beads are moved from the thermal cycler to the bead trap where they undergo washing prior to being analyzed in the flow cytometer.

The reagent delivery system 101 delivers PCR reagents to the thermal cycler 102 autonomously. On completion of cycling in the thermal cycler 102, beads or microspheres are added to the sample in the thermal cycler. The hybridized beads are then moved to the flow cytometer 104 for analyses. Alternatively, the hybridized beads are added to the bead trap 103 where they undergo washing prior to being moved to the flow cytometer.

The hybrid nucleic acid analyzer system 100 utilizes nucleic acid amplification and detection and sample preparation and analysis techniques and information described in currently co-pending U.S. patent application Ser. Nos. 10/189,319 and 10/643,797, both of which are owed by the Regents of the University of California, the assignee of this application. U.S. patent application Ser. No. 10/189,319 for an “Automated Nucleic Acid Assay System” was filed Jul. 2, 2002 by Billy W. Colston, Jr., Steve B. Brown, Shanavaz L. Nasarabadi, Phillip Belgrader, Fred Milanovich, Graham Marshall, Don Olson, and Duane Wolcott and was published as U.S. patent application No. 2003/0032172 on Feb. 13, 2003. U.S. patent application Ser. No. 10/643,797 for a “System for Autonomous Monitoring of Bioagents” was filed Aug. 19, 2003 by Richard G. Langlois, Fred Milanovich, Billy W. Colston, Jr., Steve B. Brown, Don A. Masquelier, Ray P. Mariella, and Kodomundi Venkateswaran and was published as U.S. patent application No. 2004/0038385 on Feb. 26, 2004. The disclosures of U.S. patent application Ser. Nos. 10/189,319 and 10/643,797 are incorporated herein by this reference.

A LabView interface software system controls the fluidic handling and the operation of the thermal cycler 102. The LabView interface software system software is integrated into a form compatible with the Graphical User Interface (GUI) used to control and monitor the flow cytometer 104.

Referring now to FIG. 2, additional details of the reagent delivery system 101 of the hybrid nucleic acid analyzer system 100 are shown. The reagent delivery system 101 includes a syringe pump 200 that delivers a carrier 201 to a holding coil 202. The carrier is available to a zone fluidics system. The zone fluidics system provides sequential injection analysis (SIA).

Zone fluidics defines a general-purpose fluidics tool, allowing the precise manipulation of gases, liquids and solids to accomplish very complex analytical manipulations with relatively simple hardware. Zone fluidics is the precisely controlled physical, chemical, and fluid-dynamic manipulation of zones of miscible and immiscible fluids in narrow bore conduits to accomplish sample conditioning and chemical analysis. A zone is a volume region within a flow conduit containing at least one unique characteristic.

A unit operation in zone fluidics comprises of a set of fluid handling steps intended to contribute to the transformation of the sample into a detectable species or prepare it for manipulation in subsequent unit operations. Examples of unit operations include sample filtering, dilution, enrichment, medium exchange, headspace sampling, solvent extraction, matrix elimination, de-bubbling, amplifying, hybridizing, and reacting. In current analytical practice many of these steps are handled manually or in isolated pieces of equipment. Integration is scant at best, and there is a high degree of analyst involvement. In zone fluidics, sample and reagent zones are subjected to these unit operations in a sequential manner being transported from one unit operation to the next under fluidic control.

Zone fluidics provides an alternative approach whereby unit operations are performed in narrow bore conduits and the transportation medium, instead of being mechanical as in robotics, is fluidic. At the heart of a zone fluidics manifold is a multi-position selection valve. Fluids are propelled and manipulated in the manifold by means of a bi-directional flow pump. A holding coil between the pump and valve is used to stack zones and mix adjacent zones through dispersion and diffusion as is practiced in sequential injection analysis (SIA).

The ports of the multi-position valve are coupled to various reservoirs, reactors, manifold devices, and detectors as indicated. Narrow bore conduits comprise the flow channels and provide fluid contact between manifold devices and components. The term fluid refers to liquids, gases, aerosols, and suspensions. Samples in zone fluidics are not limited to liquids. Rather, gases, and suspensions containing solids or cells are also included. Where solid samples are used, particles are limited to a size that ensures no blockages.

In most cases, reagents are prepared and then coupled to the zone fluidics manifold. The metering capability of the pump and mixing unit operations allow for reagents and standards to be prepared in situ. Reagents can therefore be presented to the zone fluidics manifold in an appropriately designed cartridge as ready-made, reagent concentrates, lyophilized, or crystalline form. Standards can be plumbed to the multi-position valve as discrete reservoirs providing the required range of concentrations. As for reagents though, standards can also be prepared in situ or diluted to cover a larger dynamic range.

In the reagent delivery system 101, a syringe pump 200 delivers a carrier 201 to a holding coil 202. The carrier is available to a zone fluidics system. The zone fluidics system provides in sequential injection analysis (SIA). The ports of a multi-position valve 203 of the zone fluidics sequential injection analysis system are coupled to air reservoir 204, negative reservoir 205, field sample reservoir 206, reagent reservoir 207, plug 208, waste 209, bleach reservoir 210, and bleach reservoir 211 as indicated. The zone fluidics sequential injection analysis system has an outlet 212 that delivers PCR reagents to the thermal cycler 102.

Referring now to FIG. 3, details of the thermal cycler 102 of the hybrid nucleic acid analyzer system 100 of the present invention are shown. Currently available Polymerase Chain Reaction (PCR) thermal cycling units are large, cumbersome and non-portable. Some examples of commercially available semi-portable instruments include the iCycler manufactured by Bio-Rad, the Light cycler from Idaho Technologies and the Smart Cycler from Cepheid Inc. In addition to amplification reactions described above, real-time PCR works by including in a reaction mix sequence specific oligonucleotides (primer) that can be extended at its 3′ end and a third non-extendable oligonucleotide (probe) that has two fluorescence molecules attached to its 5′ and 3′ end respectively. Thus the probe is quenched due to the Fluorescence Resonance Energy Transfer (FRET) between the two fluorescent molecules. FRET is dependent on the sixth power of the intermolecular separation of the two fluorophores. In the absence of primer extension, there is no fluorescence signal detected by the fluorimeter. The enzyme DNA polymerase has 5′-3′ exo-nuclease activity as well as 5′-3′ polymerase activity. During primer extension, the fluorophore is cleaved from the 5′ end of the probe and since the fluorophore is no longer quenched, a signal is detected by the fluorimeter. These instruments are designed for measuring the fluorescence released from sequence specific probes in case of a positive identification. At present the multiplexing of nucleic acid signatures is limited by the number of fluorophores that can be used in the commercial instruments, due to spectral overlap of most of these fluorophores.

The thermal cycler 102 of the present invention can be a unit such as that described in U.S. Pat. No. 5,589,136 issued Dec. 31, 1996 to M. Allen Northrup, Raymond P. Mariella, Jr., Anthony V. Carrano, and Joseph W. Balch and assigned to the Regents of the University of California or in U.S. Pat. No. 6,586,233 issued Jul. 1, 2003 to William J. Benett. James B. Richards, and Fred P. Milanovich and assigned to the Regents of the University of California. The disclosures of U.S. Pat. No. 5,589,136 issued Dec. 31, 1996 and U.S. Pat. No. 6,586,233 issued Jul. 1, 2003 are incorporated herein by this reference. In addition, the thermal cycler as set forth in U.S. application Ser. No. 10/272,178 (US PGPUB 20040072334), to Benett et al., which is expressly incorporated herein by reference, finds use in this invention. This thermal cycler is particularly useful as it the chamber units are preferably made from copper. Copper provides good thermal conductivity. This is described in more detail in US PGPUB 20040072334, which is expressly incorporated herein by reference.

The thermal-cycler allows the controlled heating and cooling of the sample to perform the PCR amplification. The thermal-cycler is a flow-through chamber made from two photo-lithographically patterned and etched copper plates. The etched channel on the inside of the chamber allows the sample tubing to be clamped between the two chamber halves, insuring good thermal contact. The tubing is connected directly to the fluidics system allowing the sample to be moved into and out of the thermal-cycler. The etched features on the outside of the chamber create increased surface area to enhance forced convective cooling.

Heating is accomplished by clamping the chamber between two circuit board assemblies. Standard surface mount resistors soldered to the circuit boards act as heaters. A surface mount linear thermistor provides temperature sensing for control of the thermal-cycling. During the cooling cycle, air is forced through slots in the circuit board assembly, onto the chamber.

As shown in FIG. 3, a chamber unit 300 is fabricated of circuit board material. The system can be constructed of materials such as circuit board fiberglass, silicon, ceramics, metal, or glass. Advantages of using circuit board fiberglass is the fact that it is not as thermally conductive as the other materials and the heating is more efficiently applied to the sample rather than being conducted to surrounding materials. Circuit board material is readily available and the technology of producing and working with circuit board material is highly developed. Circuit board material provides lower cost techniques for fabrication. Printed circuit board technology incorporates photolithography, metal etching, numerically controlled machining, and layering technologies to produce the desired device.

As shown in FIG. 3, the thermal cycler 102 is generally indicated at 300. The thermal cycler 102 includes a silicon-based sleeve as a chemical reaction chamber, generally indicated at 301, constructed of two bonded silicon parts, and which utilizes doped polysilicon for heating and bulk silicon for convective cooling, as described in greater detail hereinafter. The sleeve 301 includes a slot or opening 304 into which reaction fluid, indicated at 306, from a conduit 305 is inserted into the reaction chamber. The conduit 305 is constructed of plastic, for example, or other material which is inert with respect to the reaction mixture, thereby alleviating any potential material incompatibility issues. The sleeve is also provided with an opening 302 in which is located an optical window 303, made, for example, of silicon nitride, silicon dioxide, or polymers. The silicon sleeve reaction chamber 301 includes doped polysilicon for heating and bulk silicon for convective cooling, and combines a critical ratio of silicon and silicon nitride to the volume of material to be heated (e.g., liquid) in order to provide uniform heating, yet low power requirements.

The thermal cycler 102 can be used to rapidly and repetitively provide controlled thermal cycles to the reaction mixture. The thermal conductivity properties of the silicon or similar semiconducting substrate, help speed up the thermal rise and fall times, and allow low power operation. While silicon is unique in its thermal properties, i.e., high thermal conductivity, a combination of silicon, silicon nitride, silicon dioxide, polymers and other materials would provide a combination of thermal conductivity and insulation that would allow thermal uniformity and low power operation.

The Sample and the nucleic acid reaction mix are introduced into the thermal cycler 102 by the Sequential Injection Analysis fluid handling system illustrated in FIG. 2. As the sample is continuously driven by convection through the channels it passes through sections of channel that are temperature controlled to be at the upper and lower temperatures required for the PCR reaction. This continuous flow through the PCR temperature zones effectively thermally cycles the sample.

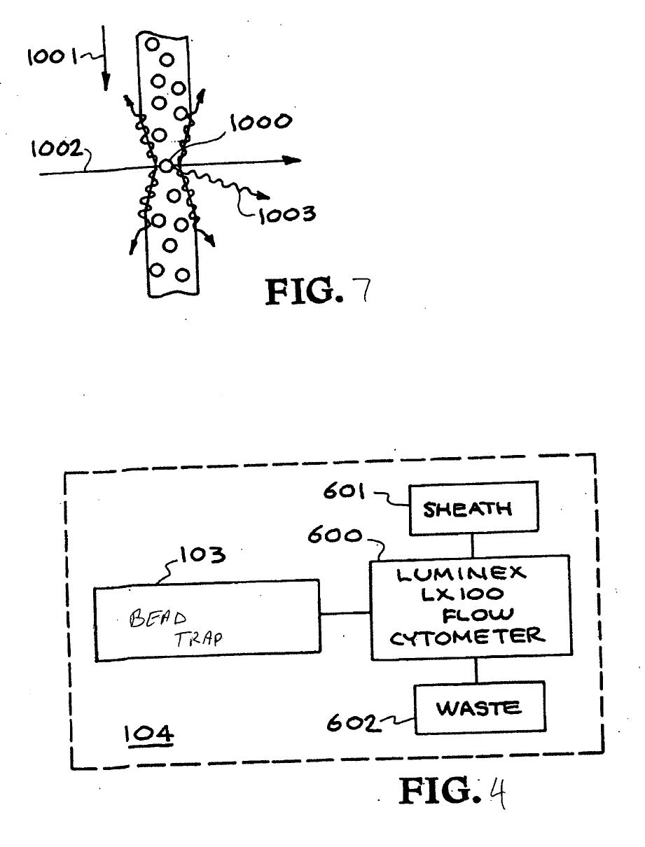

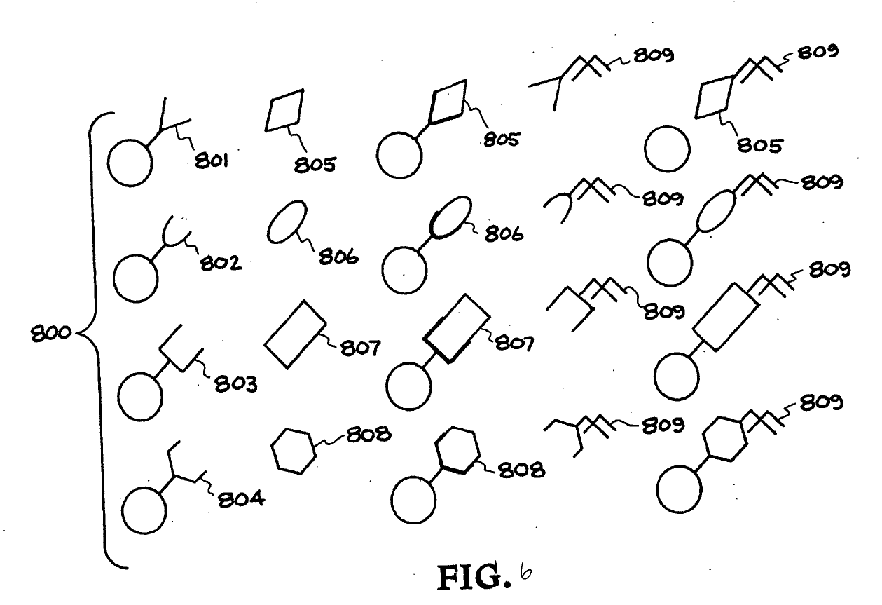

Referring now to FIG. 4, additional details of the flow cytometer 104 of the hybrid nucleic acid analyzer system 100 are shown. The flow cytometer 104 comprises a Luminex LX100 Flow Cytometer instrument 600 with a sheath source 601 and a waste reservoir 602. The hybridized bead array from the beat trap 103 is introduced into the Luminex Flow Cytometer instrument 600 where the beads are interrogated by two lasers, a red laser for the internal discriminator and a green laser for the external discriminator dyes respectively. Additional details of the flow cytometer 600 and its operation are show in FIGS. 7, 8, 9, and 10.

In order to multiplex more than four signatures, Applicants have designed an analyzer that is exemplified by the Luminex Bead based Array analyzer. With the liquid arrays it is possible to simultaneously multiplex 100 different organisms or targets. The discrimination of the polystyrene Luminex bead array is dependent on the precise ratio of two internal discriminator dyes, a red and an infrared dye. The signal intensity on the surface of the bead is dependent on the concentration of the analyte in solution, in our case the amplified DNA of a suspect agent or an antigen or a toxin, whichever the case may be.

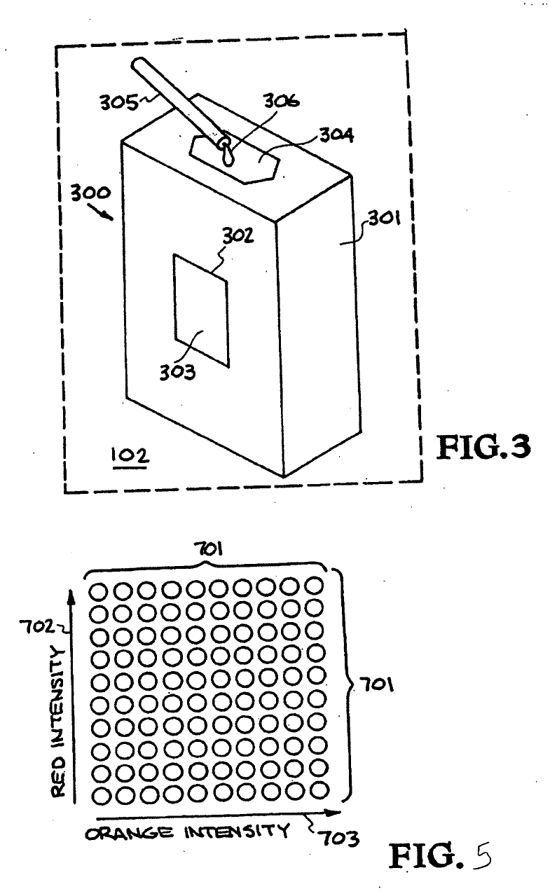

Referring now to FIG. 5, the beads of the invention are illustrated. A 100-plex Luminex liquid array 700 is generated by intercalating varying ratios of red and orange infrared dyes into polystyrene latex microspheres or beads 701. The process of producing varying ratios of red and orange infrared dyes in the beads 701 is accomplished by increasing the amount of red dye as illustrated by the arrow 702 and increasing the amount of orange dye as illustrated by the arrow 703. This gives each optically encoded bead 700 a unique spectral address.

Referring now to FIG. 6, additional information is provided illustrating how the beads are used in the flow cytometer 104. The beads designated by the reference numeral 800 are coated with capture antibodies specific for target antigens or capture probes complementary to adapter sequences as described herein. Each bead has an attachment site specific for a bioagent. For example, the upper bead has an attachment site 801 for influenza nucleic acids or a capture probe capable of hybridizing with an adapter attached to a primer specific for the influencz nucleic acids. The next bead has an attachment site 802 for an adenovirus nucleic acid or a capture probe capable of hybridizing with an adapter attached to a primer specific for the adenovirus nucleic acid, and the like.

Referring now to FIG. 7, an illustration shows how the beads are analyzed in the flow cytometer. The beads are designated by the reference numeral 1000. The direction of flow is shown by the arrow 1001. The beads 1000 are interrogated one at a time. As illustrated, one bead 1000 is shown being interrogated. A red laser classifies the bead 1000, identifying the bead type. Subsequently a green laser 1002 quantifies the assay on the bead surface—only those beads with a complete sandwich will produce a fluoresce 1003 in the green, and the signal is a function of label concentration, which is indicative of the amount of target.

The hybrid nucleic acid analyzer provides an integrated nucleic acid and protein/toxin detection system capable of in-line analysis of a complex sample within an hour or less. The hybrid nucleic acid analyzer has the capability of performing continuous nucleic acid and immunoassays in a multiplex format. The hybrid nucleic acid analyzer is a field deployable instrument for detection of pathogens and toxins in environmental or clinical samples. The hybrid nucleic acid analyzer takes advantage of the multiplexing capability of the Luminex Bead arrays complexed with multiplexed nucleic acid and protein capability developed at the Lawrence Livermore National Laboratory.

The hybrid nucleic acid analyzer has an integrated PCR chamber 102, and Luminex LX100 flow cytometer 104 and optionally a microsphere was chamber 103 controlled by a LabView interface software for the fluidic handling and the operation of the PCR chamber. The software is integrated into a form compatible with the Graphical User Interface (GUI) used to control and monitor the Luminex LX100 flow cytometer. Control and data analysis software routines have been written for controlling the Luminex LX100 flow cytometry. Provisions have been made for the addition of a sample preparation and concentration unit as well as a bead sequestering unit in order to facilitate deep multiplexing of the agents. A sample preparation and concentration strategy involves the use of Silicon pillar chips capable of handling volumes of up to 100 ml or more of the sample, releasing the DNA from the cells through lysis and concentrating it in a small volume for analysis, thus increasing the detection limit many folds.

The fluidics in the instrument is self-contained in order to minimize contamination of the surroundings and the operator. This minimizes contamination of reagents and samples, a feature not available in commercial units. The sample and the nucleic acid reaction mix are introduced into the thermal cycler 102 by Sequential Injection Analysis fluid handling system 101.

Once the sample is introduced into the instrument 100, the detection is autonomously done following the sequence of events input by the researcher. Decontamination of the fluidics system is carried out autonomously after each amplification step. The system including the PCR chamber 102, the tubing carrying the sample to the PCR chamber and all the tubing and fitting downstream from there on are rinsed with 5% household bleach which we have found sufficient to effectively remove all traces of nucleic acids or PCR product from the housing. After every PCR run, a negative control for the agent/agents is amplified in order to determine the efficacy of the decontamination process.

Manual labor is the major factor for the high cost of sample testing. The software has the capability of stacking a series of fluidic protocols for autonomous analysis. Thus the instrument 100 can be loaded with the reagents and the samples at the beginning of the day and the results can be accessed from a remote location. This cuts the cost of labor as compared to the conventional way of doing analysis. Thus with this instrument it is possible to perform continuous analysis of samples from a known set of reagents with minimal intervention in effect significantly reducing the cost of the assay.

The hybrid nucleic acid analyzer 100 provides autonomous use of both the thermal cycler 102 and the flow cytometer 104 such that protein analysis can be performed independent of the nucleic acid detection. For detection of antigens or toxins, the sample is introduced directly to appropriately labeled beads followed by hybridization to the secondary antibody and analysis of the assay in the flow cytometer 104. The hybrid nucleic acid analyzer 100 can be repeatedly decontaminated in between runs with a solution of 5% household bleach.

The nucleic acid detection is done by hybridization of the amplified PCR product with the probes attached to the surface of the bead sets, .e.g. via NHS ester linkage chemistry. The PCR product is labeled with Biotin molecules and the hybridization of the product to the beads is followed by streptavidin phycoerythrin addition to the hybridized reaction mix.

The hybrid nucleic acid analyzer system 100 provides a closed integrated rapid Real-time PCR and multiplex flow analysis instrument for identification of multiplex pathogen and toxin within an hour with minimal exposure to the technician. The hybrid nucleic acid analyzer system 100 combines Real-time flow through PCR with an inline flow cytometer to detect both nucleic acids as well as proteins. Sequential injection analysis (SIA) fluidic system is used to deliver the sample and reagent for in-line mixing, analysis and archiving of samples.

The unused PCR reaction mix is moved to the waste stream. The hybrid system is decontaminated and made ready for another round of amplification by rinsing with a 5% solution of Household Bleach followed by water rinse. A negative reaction with water substituted for sample is run between sample amplifications to ensure that the system is free of carry over PCR product.

The hybrid nucleic acid analyzer 100 has many uses. For example, the system 100 has use for clinical analysis of blood bank samples in a continuous 24/7 analysis of pathogens. The system 100 has use in diagnostic labs. The system 100 has use as a fly away lab or integrated into continuous monitoring of environmental samples for detection of Biothreat agents. The system 100 also has use in automated processing, amplification and detection of biological molecules in forensic samples. The system 100 can also be used for automated clinical testing, analysis and archiving in event of an outbreak. The system 100 can also be used to detect proteins and toxins both in the clinic as well as from the environrment.

All references cited herein are incorporated by reference in their entirety.

While the present invention has been described with reference to what are considered to be the specific embodiments, it is to be understood that the invention is not limited to such embodiments. To the contrary, the invention is intended to cover various modifications and equivalents included within the spirit and scope of the appended claims.

### EXAMPLES

### Example 1

Identification of nucleic acid signatures for multiplex amplification of respiratory pathogens

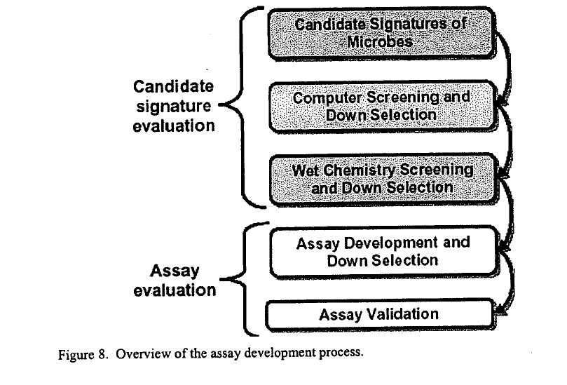

An outline of the development, optimization, and characterization of these multiplexed assays is depicted in FIG. 8. This begins with an analysis of all available genomic sequence information, which forms the basis for the development of signatures. A signature is a region or set of regions on a chromosome that is unique to that organism. The nucleic acid assays employ PCR with primer pairs to generate the signature fragment(s) of interest. Once candidate signatures were identified, they were subjected to a computational screening and down-selection process. This “in silico” screening method tested the candidate regions for uniqueness when compared to all the sequence data available. The computational screening also ensured that the signatures were amenable to assay chemistry requirements and provided a rapid, low-cost initial screening of candidate signatures. The primers that emerged from the computational screening and down-selection were then tested against an extensive panel of DNAs and cDNAs. This bench screening ensured that the primers will detect the strain diversity of the pathogen but will not react with the nucleic acids of other organisms that could be present in a sample. Primer pairs that successfully passed the wet chemistry screening criteria were advanced to the assay development stage. Assay development included the optimization of extraction and detection protocols, so that the assays perform consistently to required specifications on the prototype equipment selected.

Signature Development: Computational Methods and “in-silico” screening. A signature is a region or set of regions on a chromosome that is unique to that organism; the described work uses polymerase chain reaction (PCR) with primer pairs to generate the signature fragment(s) of interest. Signature development begins with an analysis of all available genomic sequence information compiled from both public and private databases. All available nucleic acid sequences for each pathogen were surveyed and collected. These sequences were aligned to find regions that were conserved across pathogen strains. The resulting signatures were then electronically subtracted from DNA/RNA sequence of over 13,000 types of microbes to avoid false positives from pathogen sequence matching microbia/viral sequences that constitute ‘background’. The result represents a region of the target that is both unique to that viral species and conserved across viral types. These regions were then mined for TAQMAN appropriateness (i.e.,primer/probe sequence length, relative proximity, GC content, Tm, etc). Once candidate signatures have been identified, they are subjected to a computational screening and down-selection process. This “in silico” screening method tests the candidate regions for uniqueness when compared to all the sequence data available. The computational screening also ensures that the signatures are amenable to assay chemistry requirements and provides a rapid, low-cost initial screening of candidate signatures. We generated a large number of candidate signatures from our bioinformatics survey, including 35 signatures for influenza; 119, for parainfluenza; 551 for adenovirus, 100 for respiratory syncytial virus, and 53 for SARS.

Signature Development: Wet Chemistry screening. The primers that emerged from the computational screening and down-selection were then tested against an extensive panel of DNAs and cDNAs. Background screening was conducted against DNA from soils, aerosols, microbes, zoological panels, and other sample types as appropriate. This step ensured that the primers will detect the strain diversity of the pathogen, but will not react with the nucleic acids of other organisms that could be present in a sample. The wet chemistry screening process utilizes high-throughput equipment. Hundreds to thousands of samples can be processed per day when materials are handled and maintained in a high-density format. This type of handling procedure has also enabled the electronic analysis of the data and reduced technician errors due to the tracking of such a large number samples. The data is generated as an electronic file and transferred into a relational database where the results are stored for future use and analysis. At the end of this process, we have developed the procedures that specify sample type, extraction method and the reagents for detection and instrumentation. Primer pairs that successfully pass the wet chemistry screening criteria are advanced to the assay development stage. For the respiratory assay panel under development, after removing signatures that cross-reacted with background signatures, the number of suitable candidate signatures produced by the informatics team were significantly reduced, leaving 8 signatures for influenza, 7 for parainfluenza, 8 for adenovirus, 1 for RSV, and 1 for SARS.

Taqman Assay Development for each new assay developed a gold standard assay is developed using standard Taqman assay chemistry as well. The “gold standard” enables comparison of a new assay or new assay technology against a standard assay format and instrumentation. These reagents have delivered exceptional performance for the end users, with no false positives since their deployment.

The development of gold standard assays enable assays that are precisely specified, defining the sample type, extraction protocol, detection reagents, and instrumentation. Each of the candidate signatures that emerged from the gel screening were developed into Taqman assays. Taqman assay development included optimization of all parameters and then screening against ˜2,500 background nucleic acids, and many hundreds of target and near-neighbor nucleic acids.

For this application, oligonucleotide probes with sequences that are complimentary to the target nucleic acid sequences were covalently coupled to beads. Nucleic acids from pathogens (targets) were amplified using standard PCR techniques. After target amplification, the amplicons, half of which contain the biotinylated forward (5′-3′) primer were introduced to the beads and allowed to hybridize to their complimentary probes on the appropriate bead. A fluorescent reporter molecule (strepavidin-phycoerythrin) was added, and binds the biotin functional groups within the forward primer. Therefore, the completed assay product comprises a bead+probe+biotinylated (and fluorescently tagged) amplicon. Each optically encoded and fluorescently-labeled microbead was then interrogated by the flow cytometer. The 635-nm red diode laser excites the dyes inside the bead and classifies each bead to its unique bead class, and a green “reporter” laser (532 nm) quantifies the assay at the bead surface. The flow cytometer is capable of reading several hundred beads each second; analysis can be completed in as little as 15 seconds. Conducting the assay requires multiple steps and significant thermocycling times; the process currently takes about 2 hours. These signature sequences are set forth in Table 1.

Reagents.

Covalent Coupling of Oligonucleotide Probes to COOH-microbeads:

Different sets of carboxylated fluorescent microbeads were obtained from Luminex Corp (Austin, Tex.), and probes for each assay were assigned to a unique bead set. Oligonucleotide probes, with sequences representing the reverse compliment to target region of the forward strand (5′-3′) were obtained from Integrated DNA Technologies (Coralville, Iowa). Each probe contained a C-18 spacer between the reactive group and the 5′ end of the oligo to enable optimum hybridization. Probes for each of the pathogen targets were coupled to using the manufacture's recommended coupling protocol. Briefly a 1 ml aliquot (1.25×10⁷) of beads was re-suspended in 50 μl of 0.1 M 2-[N-morpholino] ethanesulfonic acid (MES) buffer, pH 4.5. 0.05 mg of 1-ethyl-3-[3-dimethylaminopropyl]-carbodiimide hydrochloride (EDC) (Pierce Biotechnology, Rockford, Ill.) was added, along with 10 μl of probe at a concentration of 50 μM. This solution was incubated in the dark at room temperature for 30 minutes. A second aliquot of EDC (0.025 mg) was added and incubated as before. The beads were rinsed in 1 ml phosphate buffered saline (PBS) containing 0.02% Tween-20 (Sigma), centrifuged at 13,000 rpm for 2 min, rinsed in PBS containing 0.1% sodium dodocyl sulfate (SDS), centrifuged as before, and stored in 0.1 M MES buffer in the dark at 4° C. Each probe/bead conjugate was stored separately, and a fresh bead set containing all conjugates was prepared for each liquid bead array assay.

Assay Controls: Controls that convey important diagnostic information regarding reagent addition, quality and concentration, assay operator performance and instrument stability can be easily added without compromising or limiting the screening capabilities of an assay. The assays employ a unique set of five rationally-designed internal controls built into every sample that monitors and reports every step of the assay. The negative control (NC) comprises a bead coupled to a Mt7 probe. Mt7 is a nucleic acid sequence obtained from Maritima maritensis, an organism found near deep-sea thermal vents. This organism was selected to serve as a NC because it is unlikely to be observed in clinical samples. Thus, Mt7 is not expected to bind exogeneous nucleic acids and consequently, the median fluorescent intensity (MFI) of the NC beads should always be low. High MFIs on the NC beads obtained in the presence of a sample would indicate a lack of specificity. The instrument control (IC) verifies the reporter fluorescence optics of the flow analyzer. The IC is a bead to which a Cy3-labeled Mt7 probe has been coupled. The probe is unlikely to bind other nucleic acids, and the Cy3 dye emits a constant fluorescence (i.e. constant MFI) in all samples when excited by the reporter laser. A change in MFI on the IC bead indicates fluctuations in the reporter laser performance. The fluorescent control (FC) tests for the addition of the fluorescent reporter (SA-PE). FC is a bead coupled with biotinylated-Mt7 probe that fluoresces after exposure to SA-PE. A bead coupled to a specially-designed armored RNA probe serves as both a positive PCR control, as well as a control to indicate the addition of sample. Signals are obtained only when PCR product has been generated and bound to the probe, and SA-PE has been added; lack of signal on the PCR control bead indicates that either PCR was not performed properly or that SA-PE was not added. The FC control, however, will yield a signal even in the absence of PCR, so these two events can be decoupled. These controls afford high-confidence that the assay was performed correctly, by monitoring the addition of sample, confirming PCR was performed, indicating that SA-PE was added, and verifying that the instrument is performing and that the assay are specific. Every sample is analyzed in the context of the performance of the controls, thereby minimizing the likelihood of false positives.

Extraction

Extractions are conducted with a Omega Bio-Tek (Doraville, Ga) E.Z.N.A. Total RNA extraction kit. The kit is specifically designed for the simultaneous extraction of both DNA and RNA using a single procedure. Nucleic acid was extracted from deactivated antigens to use as positives when testing the various signatures. The protocol was as follows:
1—Started with 3-5 ug of antigen in 100 ul of TE.
2—Added Lysis Buffer with beta-ME and 3.75 ug of Glycogen to samples
3—Transferred mix to a Homogenization Column and centrifuged. Flow through liquid was transferred to a clean tube and 250 ul of 100% EtOH was added.
4—Transferred sample and EtOH mix to HiBind RNA spin column and centrifuged. The nucleic acid was held bound in the column matrices and the flow through was discarded.
5—The nucleic acid was washed with various buffers and then eluted in 50 ul of DEPC treated water.

Multiplexed PCR amplification: Each amplification reaction was performed in a total volume of 25 μl. The reaction mix consisted of 12.5 μl of 2×QuantiTect Probe RT-PCR Master Mix (Qiagen, Valencia, Calif.), 1 μl each of forward and reverse primers (each at a concentration of 0.4 μM), 0.25 μl per reaction of QuantiTect RT Mix, 10.25 ml of RNase-free water, and 1 μl of template. The QuantiTect Probe RT-PCR kit contains a balanced mix of KCl and NH₄(SO₂) to increase specific primer binding. The kit utilizes a mix of OmniScript and Sensiscript RT enzymes that have a high affinity and therefore are able to transcribe through secondary structures. In addition, Omniscript targets RNA>50 ng while Sensiscript targets RNA<50 ng. The Taq polymerase used is a “Hot Start” Taq that is robust and can be stored at room temp for up to two weeks. The inactive state is held by a chemical modification and not by an antibody.

Thermocycling conditions: 30 min at 50° C., 15 min at 95° C., followed by 40 cycles of 15 sec at 94° C., 30 sec at 55° C., 15 sec at 72° C., concluding with a 4° C. hold.

Hybridization: A bead set was prepared, consisting of a mixture of 25 μl of each of the covalently coupled beads listed in Table 1 into 1 ml TMAC buffer (4.5 M tetramethyl ammonium chloride, 0.15% SDS, 75 mM Tris pH 8.0, 3.0 mM EDTA pH 8.0). Amplified PCR reaction product (1 μl) was added to 22 μl of the bead mix. PCR products and bead mix were denatured at 95° C. for 2 min and allowed to hybridize at 55° C. for 5 min. The mix was transferred to a 96 well filter plate (Millipore, Bedford, Mass.). The beads were washed once in 500 μl TMAC and incubated with 60 μl of 60 ng/ml Streptavidin-phycoerythrin (SAPE) (Caltag Laboratories, Burlingame, Calif.) for 5 min. The hybridized beads were washed again with 500 μl TMAC buffer and re-suspended in a final volume of 100 μl TMAC buffer. The completed sample was then introduced to the Luminex flow analyzer for analysis.

Automated Multiplex Analysis of Respiratory Pathogens

Sample Introduction, Automated DNA Extraction, and Sample Processing

Samples are obtained as nasal or throat swab samples. The swab is placed into a tube containing 2 ml viral transport medium. The tube is capped, and shaken vigorously, in an effort to transfer viral particles from the swab into the surrounding media. The swab is then removed from the tube and discarded. The tube containing the liquid is then introduced (loaded) onto the sample port. A small volume of sample is aspirated and mixed online with the appropriate volume of each reagent required to perform RT-PCR, including a mastermix solution (RT and PCR enzymes, buffer, MgCl₂, dNTPs, BSA), primers (biotinlyated forward primer, unmodified reverse primer), and internal control (Armored RNA). These solutions are stored separately, in individual reagent vials, at room temperature on the FluIDx system (APDS ROI). The PCR reaction mixture is bracketed by air bubbles (5-10 uL) to prevent mixing with the carrier fluid as it is manipulated through the manifold tubing. The mixture is pumped into the flow-through thermal cycling chamber.

Fluidics Manifold

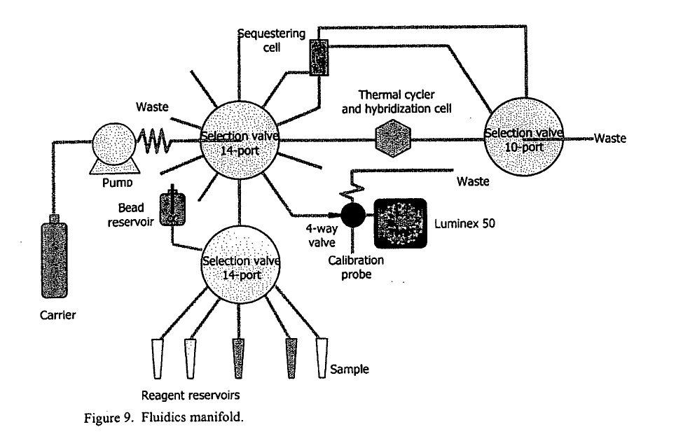

The sample preparation (fluidics) module utilizes Zone Fluidcs ²⁵ (ZF) a powerful fluidics manipulation technique that enables maximal flexibility. Zone Fluidics grew out of the more familiar fluidics technique referred to as sequential injection analysis (SIA) which has been described in detail elsewhere. Hindson, B. J.; Brown, S. B.; Marshall, G. D.; McBride, M. T.; Makarewicz, A. J.; Gutierrez, D. M.; Wolcott, D. W.; Metz, T. R.; Madabhushi, R.; Dzenitis, J. M.; and Colston, B. W. “Development of an Automated Sample Preparation Module for Environmental Monitoring of Biowarfare Agents”. Anal. Chem, 2004, 76, 3492-3497 and Hindson, B. J.; Makarewicz, A. J.; Setlur, U. S.; Henderer, B. D.; McBride. M. T.; Dzenitis, J. M. APDS: the autonomous pathogen detection system”. Biosens. Bioelectron., 2005, 20, 1925-1931. The commercially available ZF system (Flo-Pro,™ Global FIA, Fox Island, Wash.) reproduces functions routinely performed by lab personnel on the bench, allowing for sample movement, reagent addition, mixing, filtering, incubation, etc., and delivering the sample reaction volume to the assay detector. The fluidics manifold is depicted in FIG. 9. Core ZF hardware components include a bidirectional pump, a holding coil, one or more multi-position selection valves, and a computer. Various micro-analytical processors which carry out required assay unit operations are clustered around a multi-position selection valve. These include the flow-through thermal cycler used to carry out amplification of the DNA and the hybridization reaction, a bead sequestering cell to allow addition of reagents to the beads and thorough washing between additions, a stirred bead reservoir and various reagent reservoirs. The Luminex detector is also coupled to the fluid handling manifold via and interface connected to the multi-position selection valve. PFA tubing is used throughout the fluidics manifold; narrow bore tubing is used to couple the PCR reagent reservoirs to the valves. This keeps PCR reagent tube volume to a minimum which minimizes reagent waste during priming of lines. Flangeless nuts and ferrules provide tubing connections throughout the manifold.

The microbead reservoir was machined from poly(methyl methacrylate) with an internal volume and maximum fill volume of 9 and 7 mL, respectively. The stirrer shaft and paddle were stainless steel, the paddle pitch was 30°, and this assembly was coupled to a stirrer motor driven by a MC50 single channel motor speed control card (Instech Laboratories, Plymouth Meeting, Pa.). Microspheres were maintained in suspension by continuous stirring at 400 rpm. Rotation speed of the shaft was measured with an Evolution noncontact laser tachometer (Monarch Instrument, Amherst, N.H.). The exterior of the reservoir was painted black to prevent photobleaching of the microspheres.

The coaxial tubular membrane sequestering cell (Global FIA, Inc., Fox Island, Wash.) was composed of porous polypropylene with a mean internal diameter, wall thickness, pore size, and internal volume of 600 μm, 200 μm, 0.2 μm, and 26 ( 5 μL, respectively. The membrane was mounted within a shell that enabled fluid connections to selection valves to be made. Prior to use, the membrane was treated with an aqueous solution of the fluorosurfactant Zonyl FSN (0.05% v/v, Dupont) to improve wetting.

The interface to the Luminex 50 instrument comprises a four port valve (Hamilton Company, Reno Nev.). The four ports of the valve are coupled to the fluidics manifold, the Luminex 50 instrument, a holding coil which leads to waste, and a needle which can be used for introducing calibration mixes. The valve allows one of two settings. In the normal position, the Luminex, waste line, and fluidics manifold are in fluid contact. The fluidics manifold delivers the bead zone to the holding coil and the Luminex aspirates the bead zone from this coil. After the assay, the coil is flushed both by the fluidics system and by the Luminex. In the alternate position, a probe which allows the introduction of calibration mixes is coupled to the Luminex. Switching between these two positions is a manual operation.

Automated Flow-Through PCR.

The PCR thermal-cycler allows the controlled heating and cooling of the sample to perform the PCR amplification. The thermal-cycler is a flow-through chamber made from two photo-lithographically patterned and etched copper plates. The etched channel on the inside of the chamber allows the sample tubing to be clamped between the two chamber halves, insuring good thermal contact. The tubing is connected directly to the fluidics system allowing the sample to be moved into and out of the thermal-cycler. The etched features on the outside of the chamber create increased surface area to enhance forced convective cooling. Heating is accomplished by clamping the chamber between two circuit board assemblies. Standard surface mount resistors soldered to the circuit boards act as heaters. A PID temperature controller is used to control thermal-cycling and a surface mount linear thermistor provides temperature sensing for control of the thermal-cycling. During the cooling cycle, air is forced through slots in the circuit board assembly, onto the chamber. The PID temperature control requires an analog voltage set-point, an analog voltage feed back for heater temperature sense, and a digital output to turn on/off the cooling fan. The PID algorithm is handled in the main computer controller. The total thermal-cycler chamber volume using 1 mm ID tube is 23 μl, and the heating rate≈2.5°/sec, cooling rate ≈1.6°/sec.

The components of the PCR reaction are assembled by fluidics module. Typical reaction components include 25 μl enzyme master mix solution (15 μl, Accuprime Supermix I, MgCl₂ 4 mM, Invitrogen, Carlsbad, Calif.), a mixed solution of the primer pairs, and 5 μl sample. The resultant reaction mixture was positioned in the tubing of the flow-through PCR heater and subjected to a thermal cycling protocol of 95° C. for 120 s followed by 40 cycles of 95° C. for 15 s, 60° C. for 30 s, and 72° C. for 15 s. Upon completion of the PCR reaction, the amplified product is mixed with beads to which PCR reaction probes have been covalently coupled. After thermal cycling had finished, the reactor tubing was automatically cleaned with sodium hypochlorite (500 μL, 1.25% m/v) followed by a water rinse (4 mL). This cleaning procedure prevented any carryover of amplified nucleic acid and regenerated the PCR module for the next assay. All PCR reagents were stored at ambient temperature within individual reservoirs on the fluidics module. The flow-through PCR tubing was filled with deionized water when not in use.

Luminex 50 Detector

The Luminex 50 used in this work represents a custom modification of the commercially available Luminex 100 (Luminex Corp., Austin, Tex.) flow cytometer. The units is fitted with a sheath fluid delivery system (1 ml bottles used to hold an dispence clean sheath fluid (PBS+proprietary antimicrobials) and another 1 ml bottle to collect waste. Sample aspiration volume, injection rate, and analysis time were pre-determined.

Data for each sample was acquired using the Luminex 50, equipped with a high speed digital signal processor. Beads were interrogated from each bead set in the assay, and the mean fluorescence intensity (MFI) was calculated and recorded for each set; analysis was completed in 60 s for each sample.

System Control.

The system components, including the fluidics module, the PCR thermocycler, and the Luminex 50 were controlled by a Databrick computer (Datalux, Winchester, Va.) running a graphical user interface written in Labview Version 6.1 (National Instruments, Austin, Tex.). Communication between the Labview graphical user interface and the Luminex 50 was achieved using the software program Luminex LXR Library Version 2.6.9 (Luminex). Both RS-232 serial ports and data acquisition cards (PCI-6036E, National Instruments) were used for hardware communications and data transfer throughout the system.

The system software leverages developments made for two autonomous environmental monitoring systems developed at LLNL; RAIDDS and APDS. FluIDₓ uses the same drivers, step-types (script elements), routine execution engine, and most of the support architecture used in RAIDDS and APDS, including error handling and logging. However, there are differences between the software architecture used these systems. The APDS/RAIDDS software (Ver 1.x) was designed and modified to meet the highly-specialized and specific requirements of each of these two instruments. FluIDₓ will utilize the next-generation software (Version 2.x), which has been generalized such that it can be adapted to any application. Primary differences between Ver 1.x and Ver 2.x include the removal of legacy code and the addition of customization capabilities. Version 2.x software was developed with modularity in mind, which makes the changes very transparent to the user. Many of the changes involve software flow and organization.

The system will have an “Operator” screen that is distinct from either RAIDDS or APDS. The Operator screen will show the state of the system (on, off, running, where in the process the instrument is), a list of the routines that can be run, results of the previous run and have several option buttons. There will be buttons to start a run, enter data, print a report, or login for advanced operations. Note: there will be no login required for using the “Operator” level functions. Initially, there will be a login for using the “Developer” functions which include routine editing, manual operation, configuration editing, etc.

The ISDAT software is capable of handling changes to hardware with simple changes to a configuration file. This file instructs the software what port to or line to use for individual sub-systems. This allows drivers originally written for one project to be immediately useable on another project by simply indicating which port the device is now using on the new project.

The primary operational mechanism is the routine. This is a ‘script’ for executing various commands in any order desired by the designer. The routine is built and tested by an expert. The designer is able to set the order in which individual components of the system receive commands. These routines, once tested and verified, can be used by any user by simply selecting the routine and hitting “run”. The routines that are available for “operators” are selected by the designers and appear in a list on the “operator” screen. A typical list includes: main routine, cleanup, initialization, calibration, etc. This gives the user high level options without the need to understand the effects of each command in the routine.

The software already has a capability for handling algorithms with a step-type and a configuration page. These will need to be modified to handle the new algorithm being developed. The person doing the new algorithm knows the form the algorithm needs to be in to function within the ISDAT code.

The ISDAT code is setup to communicate with outside entities. The preferred mechanism is via Ethernet. This is the most reliable and readily available communications media. This allows for wireless (WiFi, or cellular), wired (any Ethernet will do) or even within the same computer. The APDS communications system uses Virtual Private Networking (VPN) and a customized low bandwidth protocol to communicate with its central data server.

Signal Analysis.

During analysis, the detector counts the instances a given bead appears in the course of that sample analysis. The detector records the fluorescent intensity of each classified and counted bead. Thus, each bead type will have a distribution of fluorescence values associated with it. To quantify the response of each bead type in an individual assay, the distribution of fluorescence is summarized by its median value. Thus, each assay results in median fluorescent intensity (MFI) value for each bead type within the assay. The results of the assay are evaluated (deemed positive or negative) based upon these MFI signals.

The interpretation of the data begins with a determination of the validity of the data by evaluating the MFI values of the internal assay controls. Each control is evaluated with respect to a MFI value which is pre-determined based on assays run during the development of the instrument. If the MFI values for the control beads fall below this predetermined value (or in the case of a negative control, rise above the pre-determined value), the results of the assay are suspect, and thus, the MFI signals cannot be evaluated with respect to determining whether a given signal is positive or negative. If all of the controls are within their acceptable ranges, a call of positive or negative can be made.

To interpret the MFI values with respect to being positive or negative, each signal from each bead type is again compared to a threshold value. These threshold values are determined by establishing a rate at which blank samples are correctly ruled as a negative. This requires measuring the MFI of each bead type for a large number (˜1000) of blank samples and generating a histogram describing the distribution of the resulting MFI values for each individual signature. From each histogram, a function can be derived describing the relationship between a threshold value and the rate at which false positives occur. After an acceptable rate of false positives is selected, the MFI threshold for each bead type can then be determined from this function. When during the course of an assay, a bead reports an MFI signal above its associated threshold value, that assay would be ruled positive, otherwise the assay is ruled negative.

What is claimed is:

1. A composition comprising the nucleic acids of SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: SEQ ID NO: 28, SEQ ID NO: 29, SEQ ID NO: 31, SEQ ID NO: 32, SEQ ID NO: 34, SEQ ID NO: 35, SEQ ID NO: 37, SEQ ID NO: 38, SEQ ID NO: 40, SEQ ID NO: 41, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 67, SEQ ID NO: 68, SEQ ID NO: 70, SEQ ID NO: 71, SEQ ID NO: 73, and SEQ ID NO: 74.

2. A method of detecting a pathogen from a sample comprising:
a) contacting said sample with the primer pairs of claim 1, wherein a first nucleic acid of each of said primer pairs comprises a target specific sequence and an adapter and a second of each of said primer pairs comprises a target specific sequence;
b) performing an amplification whereby a pathogen specific sequence is amplified;
c) contacting said amplified pathogen specific sequence with an immobilized capture probe complementary to said adapter; and
d) detecting the presence of said pathogen specific sequence.

3. The method of claim 2, wherein said amplification is performed by a method selected from the group consisting of PCR, LCR, TaqMan PCR.

4. The method of claim 2, wherein said capture probe is immobilized to a solid support selected from the group consisting of microspheres and planar arrays.

5. The method of claim 4, wherein when said solid support is microsphere, said detecting comprises applying said microspheres to a flow cytometer.

6. The method of claim 5, wherein said microsphere comprises a label that identifies its capture probe.

7. The method of claim 4, wherein when said solid support is a planar array said capture probes are at determinable positions and said method comprises detecting said pathogen specific sequence at a location on said array.

8. The method of claim 2, wherein at least one primer of each of said primer pairs comprises a label.

9. The method of claim 8, wherein said label is a primary label.

10. The method of claim 9, wherein said label is a secondary label.

11. The method of claim 10, wherein said secondary label is biotin and said method further comprises contacting said biotin with labeled avidin.

12. The method of claim 8, 9, 10 or 11, wherein said label is a fluorescent label.

13. A kit for the detection of a first target nucleic acid sequence comprising:
a) the primer pairs of claim 1, wherein a first nucleic acid of each of said primer pairs comprises a target specific sequence and an adapter and a second of each of said primer pairs comprises a target specific sequence and wherein at least one of said primers comprises a label;
b) at least a first amplification enzyme; and
c) immobilized capture probes.

14. The kit according to claim 13, wherein a first capture probe is immobilized to a first subpopulation of microspheres and a second capture probe is immobilized to a second subpopulation of microspheres.

15. The kit according to claim 13, wherein a first capture probe is immobilized at a first position on a planar substrate and a second capture probe is immobilized at a second position on a planar substrate.

16. The kit according to claim 13 for the detection of a PCR reaction wherein said first enzyme is a thermostable DNA polymerase.

17. A composition comprising the nucleic acids of SEQ ID NO: 3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, SEQ ID NO:30, SEQ ID NO:33, SEQ ID NO:36, SEQ ID NO:39, SEQ ID NO:42, SEQ ID NO:45, SEQ ID NO:48, SEQ ID NO:51, SEQ ID NO:54, SEQ ID NO:57, SEQ ID NO:60, SEQ ID NO:63, SEQ ID NO:66, SEQ ID NO:69, SEQ ID NO:72 and SEQ ID NO:75.


SEQUENCE LISTING (75 sequences)

SEQ ID NO: 1 - DNA, 23 bp, Artificial Sequence
  Description of Artificial Sequence Synthetic oligonucleotide
agcgtcaaaaatgggacttatga

SEQ ID NO: 2 - DNA, 23 bp, Artificial Sequence
  Description of Artificial Sequence Synthetic oligonucleotide
aaagacccattagagcacatcca

SEQ ID NO: 3 - DNA, 27 bp, Artificial Sequence
  Description of Artificial Sequence Synthetic oligonucleotide
tggcgatctattcaactgtcgccagtt

SEQ ID NO: 4 - DNA, 24 bp, Artificial Sequence
  Description of Artificial Sequence Synthetic oligonucleotide
ctttcagctacagatgcagacaca

SEQ ID NO: 5 - DNA, 22 bp, Artificial Sequence
  Description of Artificial Sequence Synthetic oligonucleotide
ttcccattgtgactgtcctcaa

SEQ ID NO: 6 - DNA, 29 bp, Artificial Sequence
  Description of Artificial Sequence Synthetic oligonucleotide
cgaacaattcaaccgacactgttgacaca

SEQ ID NO: 7 - DNA, 24 bp, Artificial Sequence
  Description of Artificial Sequence Synthetic oligonucleotide
gccattaacgggattacaaacaag

SEQ ID NO: 8 - DNA, 27 bp, Artificial Sequence
  Description of Artificial Sequence Synthetic oligonucleotide
ccagtagaaccaacaattctgcattat

SEQ ID NO: 9 - DNA, 32 bp, Artificial Sequence
  Description of Artificial Sequence Synthetic oligonucleotide
tcgagaaaatgaacactcaattcacagctgtg

SEQ ID NO: 10 - DNA, 21 bp, Artificial Sequence
  Description of Artificial Sequence Synthetic oligonucleotide
atgctgaggatatgggcaatg

SEQ ID NO: 11 - DNA, 24 bp, Artificial Sequence
  Description of Artificial Sequence Synthetic oligonucleotide
gatatggcaaaggaaatccatagg

SEQ ID NO: 12 - DNA, 31 bp, Artificial Sequence
  Description of Artificial Sequence Synthetic oligonucleotide
cattaaacaaccggttccagatcaaaggtgt

SEQ ID NO: 13 - DNA, 25 bp, Artificial Sequence
  Description of Artificial Sequence Synthetic oligonucleotide
tatcacaaatgtgataatgcatgca

SEQ ID NO: 14 - DNA, 27 bp, Artificial Sequence
  Description of Artificial Sequence Synthetic oligonucleotide
atgaaacccaatagaacaacacaaatt

SEQ ID NO: 15 - DNA, 29 bp, Artificial Sequence
  Description of Artificial Sequence Synthetic oligonucleotide
tgtggatttcattcgccatatcatgcttc

SEQ ID NO: 16 - DNA, 23 bp, Artificial Sequence
  Description of Artificial Sequence Synthetic oligonucleotide
gggaggaaatagacggagtcaaa

SEQ ID NO: 17 - DNA, 23 bp, Artificial Sequence
  Description of Artificial Sequence Synthetic oligonucleotide
tagatgcaaattctgcactgcaa

SEQ ID NO: 18 - DNA, 27 bp, Artificial Sequence
  Description of Artificial Sequence Synthetic oligonucleotide
tcaacagtggcgagttccctagcactg

SEQ ID NO: 19 - DNA, 24 bp, Artificial Sequence
  Description of Artificial Sequence Synthetic oligonucleotide
gtatgggtaccaccatagcaatga

SEQ ID NO: 20 - DNA, 25 bp, Artificial Sequence
  Description of Artificial Sequence Synthetic oligonucleotide
tgttcattttgtcaatgatcgagtt

SEQ ID NO: 21 - DNA, 29 bp, Artificial Sequence
  Description of Artificial Sequence Synthetic oligonucleotide
tgcagacaaagaatccactcaaaaggcaa

SEQ ID NO: 22 - DNA, 26 bp, Artificial Sequence
  Description of Artificial Sequence Synthetic oligonucleotide
gatctaaatggagtgaagcctctcat

SEQ ID NO: 23 - DNA, 23 bp, Artificial Sequence
  Description of Artificial Sequence Synthetic oligonucleotide
tatgtaagaccattccggcacat

SEQ ID NO: 24 - DNA, 27 bp, Artificial Sequence
  Description of Artificial Sequence Synthetic oligonucleotide
ctggatggctcctcggaaaccctatgt

SEQ ID NO: 25 - DNA, 27 bp, Artificial Sequence
  Description of Artificial Sequence Synthetic oligonucleotide
gacaatgaatgtatggaaagtgtgaga

SEQ ID NO: 26 - DNA, 27 bp, Artificial Sequence
  Description of Artificial Sequence Synthetic oligonucleotide
atccaaaaagatagaccagctatcatg

SEQ ID NO: 27 - DNA, 26 bp, Artificial Sequence
  Description of Artificial Sequence Synthetic oligonucleotide
cagtggcaagttccctagcactggca

SEQ ID NO: 28 - DNA, 23 bp, Artificial Sequence
  Description of Artificial Sequence Synthetic oligonucleotide
gatcccaatgacacagtgacctt

SEQ ID NO: 29 - DNA, 22 bp, Artificial Sequence
  Description of Artificial Sequence Synthetic oligonucleotide
ttccccacagttctagggttga

SEQ ID NO: 30 - DNA, 30 bp, Artificial Sequence
  Description of Artificial Sequence Synthetic oligonucleotide
catagcccctgacagggcaagtttctttag

SEQ ID NO: 31 - DNA, 27 bp, Artificial Sequence
  Description of Artificial Sequence Synthetic oligonucleotide
ggctacaaagatgtgatactttggttt

SEQ ID NO: 32 - DNA, 24 bp, Artificial Sequence
  Description of Artificial Sequence Synthetic oligonucleotide
catgtttccattcttcacacacat

SEQ ID NO: 33 - DNA, 22 bp, Artificial Sequence
  Description of Artificial Sequence Synthetic oligonucleotide
cttctggccattgcaatgggcc

SEQ ID NO: 34 - DNA, 22 bp, Artificial Sequence
  Description of Artificial Sequence Synthetic oligonucleotide
taagcagcggctacaaagatgt

SEQ ID NO: 35 - DNA, 19 bp, Artificial Sequence
  Description of Artificial Sequence Synthetic oligonucleotide
gcaccgcatgtttccattt

SEQ ID NO: 36 - DNA, 30 bp, Artificial Sequence
  Description of Artificial Sequence Synthetic oligonucleotide
catgtttcatacttctggccattgcaatgg

SEQ ID NO: 37 - DNA, 22 bp, Artificial Sequence
  Description of Artificial Sequence Synthetic oligonucleotide
aatgctatcaccaatgcgaaaa

SEQ ID NO: 38 - DNA, 23 bp, Artificial Sequence
  Description of Artificial Sequence Synthetic oligonucleotide
aacgtgtagctgtatgcttccaa

SEQ ID NO: 39 - DNA, 36 bp, Artificial Sequence
  Description of Artificial Sequence Synthetic oligonucleotide
tgacaataaaggagcattcaaatatatcaagccaca

SEQ ID NO: 40 - DNA, 20 bp, Artificial Sequence
  Description of Artificial Sequence Synthetic oligonucleotide
cgctttcacagtccaactgc

SEQ ID NO: 41 - DNA, 20 bp, Artificial Sequence
  Description of Artificial Sequence Synthetic oligonucleotide
gctgcttgtgggtttgatga

SEQ ID NO: 42 - DNA, 31 bp, Artificial Sequence
  Description of Artificial Sequence Synthetic oligonucleotide
cgttttcggattatgattcccatcgttcttc

SEQ ID NO: 43 - DNA, 20 bp, Artificial Sequence
  Description of Artificial Sequence Synthetic oligonucleotide
tcctgcaccattcccagata

SEQ ID NO: 44 - DNA, 20 bp, Artificial Sequence
  Description of Artificial Sequence Synthetic oligonucleotide
cctccgggacctgtttgtaa

SEQ ID NO: 45 - DNA, 31 bp, Artificial Sequence
  Description of Artificial Sequence Synthetic oligonucleotide
cagctttccagccttgaattattcgtgtcag

SEQ ID NO: 46 - DNA, 23 bp, Artificial Sequence
  Description of Artificial Sequence Synthetic oligonucleotide
agcgcgtaatatttgtctagggc

SEQ ID NO: 47 - DNA, 27 bp, Artificial Sequence
  Description of Artificial Sequence Synthetic oligonucleotide
tcagctgactataataataaaacgcca

SEQ ID NO: 48 - DNA, 26 bp, Artificial Sequence
  Description of Artificial Sequence Synthetic oligonucleotide
cggaacgcggaaaacacctgagaaaa

SEQ ID NO: 49 - DNA, 20 bp, Artificial Sequence
  Description of Artificial Sequence Synthetic oligonucleotide
tcgatcttacctgccacgag

SEQ ID NO: 50 - DNA, 22 bp, Artificial Sequence
  Description of Artificial Sequence Synthetic oligonucleotide
gccacaggtcctcatatagcaa

SEQ ID NO: 51 - DNA, 33 bp, Artificial Sequence
  Description of Artificial Sequence Synthetic oligonucleotide
tgctccacataatctaacacaaactcctcaccc

SEQ ID NO: 52 - DNA, 21 bp, Artificial Sequence
  Description of Artificial Sequence Synthetic oligonucleotide
cccaacacctagcctaaagcc

SEQ ID NO: 53 - DNA, 22 bp, Artificial Sequence
  Description of Artificial Sequence Synthetic oligonucleotide
tttccaagacatcttccagtcg

SEQ ID NO: 54 - DNA, 26 bp, Artificial Sequence
  Description of Artificial Sequence Synthetic oligonucleotide
aagtcaccagactcgcgctttaggcc

SEQ ID NO: 55 - DNA, 20 bp, Artificial Sequence
  Description of Artificial Sequence Synthetic oligonucleotide
tggtccagatggaaaggtca

SEQ ID NO: 56 - DNA, 21 bp, Artificial Sequence
  Description of Artificial Sequence Synthetic oligonucleotide
cttttgctgttgcctctgtca

SEQ ID NO: 57 - DNA, 35 bp, Artificial Sequence
  Description of Artificial Sequence Synthetic oligonucleotide
tgtcacacttacaccctaacttatacccaggctca

SEQ ID NO: 58 - DNA, 25 bp, Artificial Sequence
  Description of Artificial Sequence Synthetic oligonucleotide
gcgttctgattagcatagtcacact

SEQ ID NO: 59 - DNA, 24 bp, Artificial Sequence
  Description of Artificial Sequence Synthetic oligonucleotide
gcatttgtatgcagtaacattcca

SEQ ID NO: 60 - DNA, 33 bp, Artificial Sequence
  Description of Artificial Sequence Synthetic oligonucleotide
ttgtccatgtagtttgtggataagtcccattca

SEQ ID NO: 61 - DNA, 19 bp, Artificial Sequence
  Description of Artificial Sequence Synthetic oligonucleotide
gcatcggcactctccagtt

SEQ ID NO: 62 - DNA, 20 bp, Artificial Sequence
  Description of Artificial Sequence Synthetic oligonucleotide
caccatgggacattcaatcg

SEQ ID NO: 63 - DNA, 31 bp, Artificial Sequence
  Description of Artificial Sequence Synthetic oligonucleotide
agttcactccctcggtctacttcaacccctt

SEQ ID NO: 64 - DNA, 20 bp, Artificial Sequence
  Description of Artificial Sequence Synthetic oligonucleotide
tgcaattttgttgggtttcg

SEQ ID NO: 65 - DNA, 21 bp, Artificial Sequence
  Description of Artificial Sequence Synthetic oligonucleotide
cctggctgttattttccacca

SEQ ID NO: 66 - DNA, 31 bp, Artificial Sequence
  Description of Artificial Sequence Synthetic oligonucleotide
ttaatcatggttcttcctgttcttccctccc

SEQ ID NO: 67 - DNA, 20 bp, Artificial Sequence
  Description of Artificial Sequence Synthetic oligonucleotide
tggctaattgcattgcatcc

SEQ ID NO: 68 - DNA, 20 bp, Artificial Sequence
  Description of Artificial Sequence Synthetic oligonucleotide
ctcgtccccttttattggca

SEQ ID NO: 69 - DNA, 31 bp, Artificial Sequence
  Description of Artificial Sequence Synthetic oligonucleotide
acatgcgggacaaacagaataccagtgaatc

SEQ ID NO: 70 - DNA, 21 bp, Artificial Sequence
  Description of Artificial Sequence Synthetic oligonucleotide
tgtcaagtaattgcggaagca

SEQ ID NO: 71 - DNA, 26 bp, Artificial Sequence
  Description of Artificial Sequence Synthetic oligonucleotide
gccaatttgactcatagtaagcaatg

SEQ ID NO: 72 - DNA, 35 bp, Artificial Sequence
  Description of Artificial Sequence Synthetic oligonucleotide
aagacaactccgttttccttcattagagtacctgc

SEQ ID NO: 73 - DNA, 23 bp, Artificial Sequence
  Description of Artificial Sequence Synthetic oligonucleotide
caacggaatgctgttcaatacaa

SEQ ID NO: 74 - DNA, 24 bp, Artificial Sequence
  Description of Artificial Sequence Synthetic oligonucleotide
tcttctagatctgatttggccttg

SEQ ID NO: 75 - DNA, 33 bp, Artificial Sequence
  Description of Artificial Sequence Synthetic oligonucleotide
tgagctcgattgatatgtcaattggatcaagtg